(19) United States
(12) Patent Application Publication
(10) Pub. No.: US 2023/0109287 A1
(43) Pub. Date: Apr. 6, 2023

(54) COMPOUND, NON-LINEAR OPTICAL MATERIAL, RECORDING MEDIUM, METHOD FOR RECORDING INFORMATION, AND METHOD FOR READING INFORMATION

(71) Applicant: Panasonic Intellectual Property Management Co., Ltd., Osaka (JP)

(72) Inventors: MASAKO YOKOYAMA, Osaka (JP); NAOYA SAKATA, Hyogo (JP); KENJI TAGASHIRA, Osaka (JP); KOTA ANDO, Osaka (JP)

(21) Appl. No.: 18/055,313

(22) Filed: Nov. 14, 2022

Related U.S. Application Data
(63) Continuation of application No. PCT/JP2021/015827, filed on Apr. 19, 2021.

(30) Foreign Application Priority Data
Jun. 1, 2020 (JP) 2020-095348

(51) Int. Cl. G11B 7/25; C07D 251/24; G11B 7/0045

(52) U.S. Cl. CPC G11B 7/25; C07D 251/24; G11B 7/0045

(57) ABSTRACT

A non-linear optical material is represented by formula (1) below. In formula (1), L¹ to L³ are each independently represented by formula (2) or (3) below.

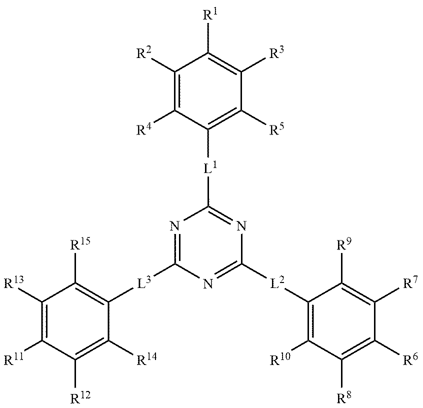

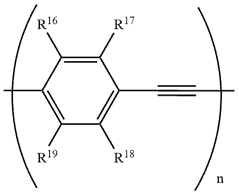

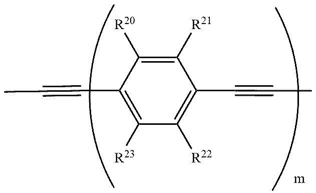

### BACKGROUND

### 1. Technical Field

The present disclosure relates to a compound, a non-linear optical material, a recording medium, a method for recording information, and a method for reading information.

### 2. Description of the Related Art

Among optical materials, such as light-absorbing materials, materials having a non-linear optical effect are referred to as non-linear optical materials. The non-linear optical effect is an effect in which when intense light, such as laser light, is projected onto a substance, an optical phenomenon occurs in the substance in proportion to the square or a higher power of the electric field of the projected light. Examples of the optical phenomenon include absorption, reflection, scattering, and emission. Examples of second-order non-linear optical effects, which are produced in proportion to the square of the electric field of projected light, include second harmonic generation (SHG), the Pockels effect, and the parametric effect. Examples of third-order non-linear optical effects, which are produced in proportion to the cube of the electric field of projected light, include two-photon absorption, multi-photon absorption, third harmonic generation (THG), and the Kerr effect.

Many studies have been actively conducted on non-linear optical materials to date. Non-linear optical materials that have been particularly developed are inorganic materials, from which single crystals can be easily prepared. In recent years, the development of a non-linear optical material made of an organic material has been expected. Compared with inorganic materials, organic materials provide high design flexibility and, in addition, have a large non-linear optical constant. Furthermore, in organic materials, a non-linear response takes place rapidly. In this specification, a non-linear optical material including an organic material may be referred to as an organic non-linear optical material.

### SUMMARY

In one general aspect, the techniques disclosed here feature a non-linear optical material represented by formula (1) below.

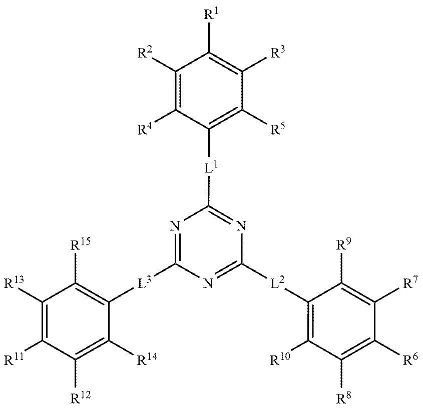

In formula (1), R¹ to R¹⁵ each independently include at least one atom selected from the group consisting of H, C, N, O, F, P, S, C1, I, and Br, and L¹ to L³ are each independently represented by formula (2) or (3) below.

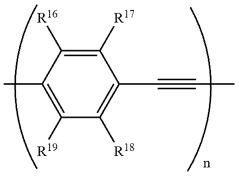

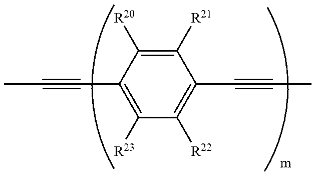

In formula (2), R¹⁶ to R¹⁹ each independently include at least one atom selected from the group consisting of H, C, N, O, F, P, S, C1, I, and Br, and n is an integer of 1 to 3. In formula (3), R²⁰ to R²³ each independently include at least one atom selected from the group consisting of H, C, N, O, F, P, S, C1, I, and Br, and m is an integer of 1 to 3. When the non-linear optical material is represented by formula (4) below, at least one selected from the group consisting of R¹, R⁶, and R¹¹ is a halogen atom, a halogenated alkyl group, an unsaturated hydrocarbon group, a hydroxyl group, an alkoxycarbonyl group, an acyl group, an amide group, an acyloxy group, a thiol group, an alkylthio group, a sulfonic acid group, an acylthio group, an alkylsulfonyl group, a sulfonamide group, a primary amino group, a secondary amino group, or a nitro group.

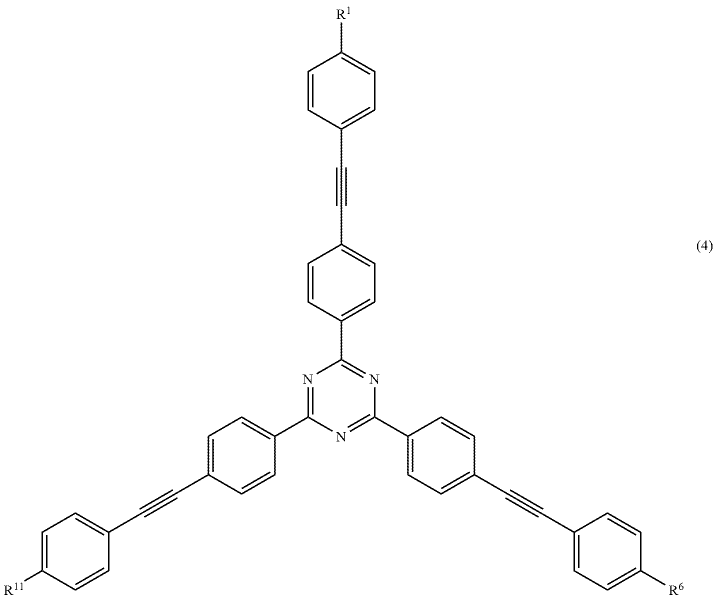

It should be noted that general or specific embodiments may be implemented as a system, a method, an integrated circuit, a computer program, a storage medium, or any selective combination thereof.

Additional benefits and advantages of the disclosed embodiments will become apparent from the specification and drawings. The benefits and/or advantages may be individually obtained by the various embodiments and features of the specification and drawings, which need not all be provided in order to obtain one or more of such benefits and/or advantages.

### BRIEF DESCRIPTION OF THE DRAWINGS

FIG. 1A is a flowchart illustrating a method for recording information, which is a method using a recording medium that includes a compound according to an embodiment of the present disclosure;

FIG. 1B is a flowchart illustrating a method for reading information, which is a method using a recording medium that includes a compound according to an embodiment of the present disclosure,

FIG. 2 is a graph illustrating a ¹H-NMR spectrum of compound (6)-7;

FIG. 3 is a graph illustrating a ¹H-NMR spectrum of compound (6)-9;

FIG. 4 is a graph illustrating a ¹H-NMR spectrum of compound (6)-10, and

FIG. 5 is a graph illustrating a ¹H-NMR spectrum of compound (7)-7.

### DETAILED DESCRIPTION

A need exists for a novel compound or a non-linear optical material that has two-photon absorption properties with respect to light having a wavelength in a short wavelength range.

The present disclosure provides a novel compound or a non-linear optical material that has two-photon absorption properties with respect to light having a wavelength in a short wavelength range.

### Underlying Knowledge Forming Basis of the Present Disclosure

Organic non-linear optical materials that are particularly attracting attention are two-photon absorption materials. Two-photon absorption is a phenomenon in which a compound absorbs two photons nearly simultaneously and enters an excited state. Two-photon absorption in a wavelength range in which no single-photon absorption band exists is referred to as non-resonant two-photon absorption. On the other hand, two-photon absorption in which a compound absorbs a first photon and thereafter further absorbs a second photon to enter a higher level of excited state is referred to as resonant two-photon absorption. In resonant two-photon absorption, a compound absorbs two photons sequentially.

In non-resonant two-photon absorption, an amount of light absorbed by a compound is usually proportional to the square of an intensity of projected light, that is, is non-linear. The amount of absorbed light can be utilized as an index of efficiency of two-photon absorption. When the amount of light absorbed by a compound is non-linear, it is possible, for example, to cause the absorption of light by the compound to occur only at or near a focal point of a laser having a high electric field strength. That is, in a sample including a two-photon absorption material, excitation of a compound only at a desired position can be realized. As such, compounds in which non-resonant two-photon absorption can occur provide very high spatial resolution, and, therefore, application of such compounds to a recording layer of a three-dimensional optical memory, a photocurable resin composition for stereolithography, and the like is being studied. When a two-photon absorption material further has a fluorescence property, the two-photon absorption material can also be utilized in fluorochrome materials that are used in two-photon fluorescence microscopes and the like. Using such a two-photon absorption material in a three-dimensional optical memory makes it possible to employ a method of reading the ON/OFF state of a recording layer based on changes in the fluorescence from the two-photon absorption material. Currently used optical memories employ a method of reading the ON/OFF state of a recording layer based on changes in a reflectance of light and an absorptance of light in a light-absorbing material.

Many organic two-photon absorbing materials having a large two-photon absorption cross section have been proposed to date. The two-photon absorption cross section is an index indicating efficiency of two-photon absorption. The two-photon absorption cross section is expressed in the units of GM (10⁻⁵⁰ cm⁴ s -molecule⁻¹ -photon'⁻¹). Many compounds having a large two-photon absorption cross section of approximately greater than 500 GM have been reported to date (e.g., Harry L. Anderson et al., “Two-Photon Absorption and the Design of Two-Photon Dyes”, Angew. Chem. Int. Ed. 2009, Vol. 48, pp. 3244-3266). However, in most of the reports, the two-photon absorption cross section is measured by using laser light having a wavelength longer than 600 nm. In some cases, the laser light used is near-infrared light, which has a wavelength longer than 750 nm.

However, applying a two-photon absorbing material to industrial uses requires that the material exhibit a large two-photon absorption cross section when laser light having a shorter wavelength is projected onto the material. For example, in the field of three-dimensional optical memories, laser light having a short wavelength realizes a finer focal spot and, therefore, improves the recording density of three-dimensional optical memories. In addition, in the field of stereolithography, laser light having a short wavelength realizes higher-resolution additive manufacturing. In particular, the standardized Blu-ray (registered trademark) discs use laser light having a center wavelength of 405 nm. Accordingly, developing a compound having a large two-photon absorption cross section with respect to light in the same wavelength range as the laser light can significantly contribute to the advancement of the industry.

Japanese Patent No. 5769151 discloses a compound having a large two-photon absorption cross section with respect to light having a wavelength of approximately 405 nm. Japanese Patent No. 5821661 and Japanese Unexamined Patent Application Publication No. 2013-242939 each disclose a compound included in an optical information recording medium in which the write time can be reduced when laser light having a wavelength of approximately 405 nm is used.

Japanese Patent No. 5769151 describes a benzene derivative having a structure with an extended π electron conjugated system. In this benzene derivative, as a result of the extension of the π electron conjugated system, the two-photon absorption cross section is increased, but a single-photon absorption peak is shifted to a longer wavelength range. Consequently, a portion of the wavelength range in which the single-photon absorption peak occurs overlaps the wavelength of excitation light. The wavelength of the excitation light is, for example, 405 nm as specified by the Blu-ray (registered trademark) standard. If single-photon absorption is caused by excitation light, the non-linearity of two-photon absorption decreases in the compound. The decrease in the non-linearity of two-photon absorption presents a significant problem, for example, in multi-layering a recording layer of a three-dimensional optical memory.

The present inventors diligently performed studies and, consequently, newly discovered that a compound represented by formula (1), described below, has excellent two-photon absorption properties and low single-photon absorption properties, with respect to light having a wavelength in a short wavelength range. In the present specification, the “short wavelength range” is a wavelength range including a wavelength of 405 nm and is, for example, a wavelength range of greater than or equal to 390 nm and less than or equal to 420 nm. In particular, the compound represented by formula (1) has a large two-photon absorption cross section with respect to light having a wavelength of approximately 405 nm. In addition, the compound has a low single-photon absorbance with respect to light having a wavelength of approximately 405 nm. In other words, the compound has two-photon absorption properties in which high non-linearity is exhibited with respect to light having a wavelength of approximately 405 nm.

### Overview of Aspects of the Present Disclosure

According to a first aspect of the present disclosure, a non-linear optical material is represented by formula (1) below,

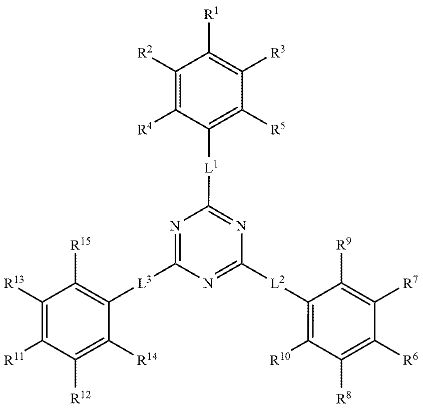

where R¹ to R¹⁵ each independently include at least one atom selected from the group consisting of H, C, N, O, F, P, S, C1, I, and Br, and L¹ to L³ are each independently represented by formula (2) or (3) below,

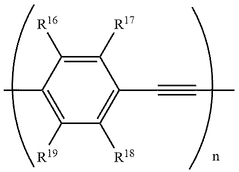

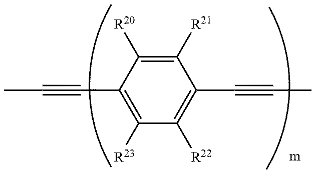

where
R¹⁶ to R¹⁹ each independently include at least one atom selected from the group consisting of H, C, N, O, F, P, S, C1, I, and Br, and n is an integer of 1 to 3, and
R²⁰ to R²³ each independently include at least one atom selected from the group consisting of H, C, N, O, F, P, S, C1, I, and Br, and m is an integer of 1 to 3,
wherein, when the non-linear optical material is represented by formula (4) below, at least one selected from the group consisting of R¹, R⁶, and R¹¹ is a halogen atom, a halogenated alkyl group, an unsaturated hydrocarbon group, a hydroxyl group, an alkoxycarbonyl group, an acyl group, an amide group, an acyloxy group, a thiol group, an alkylthio group, a sulfonic acid group, an acylthio group, an alkylsulfonyl group, a sulfonamide group, a primary amino group, a secondary amino group, or a nitro group.

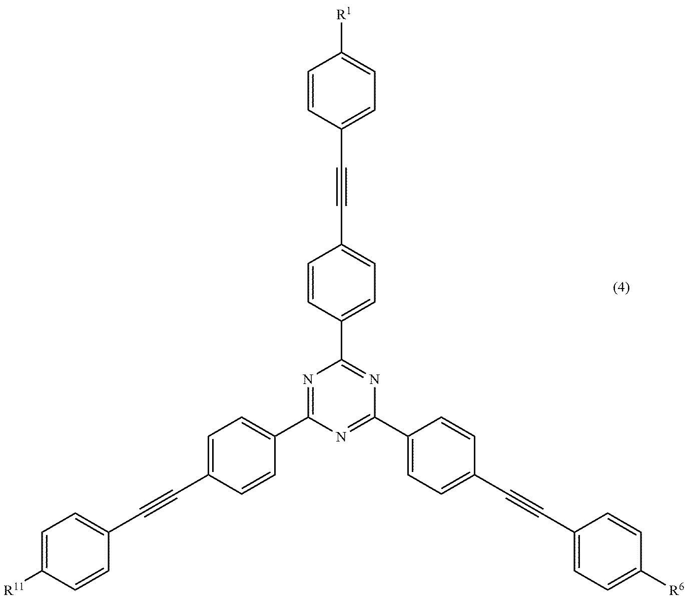

With regard to the first aspect, the non-linear optical material has excellent two-photon absorption properties and low single-photon absorption properties, with respect to light having a wavelength in a short wavelength range. That is, the non-linear optical material has two-photon absorption properties in which high non-linearity is exhibited with respect to light having a wavelength in a short wavelength range.

In a second aspect of the present disclosure, the non-linear optical material according to the first aspect may be one in which, for example, the non-linear optical material is represented by formula (5) below,

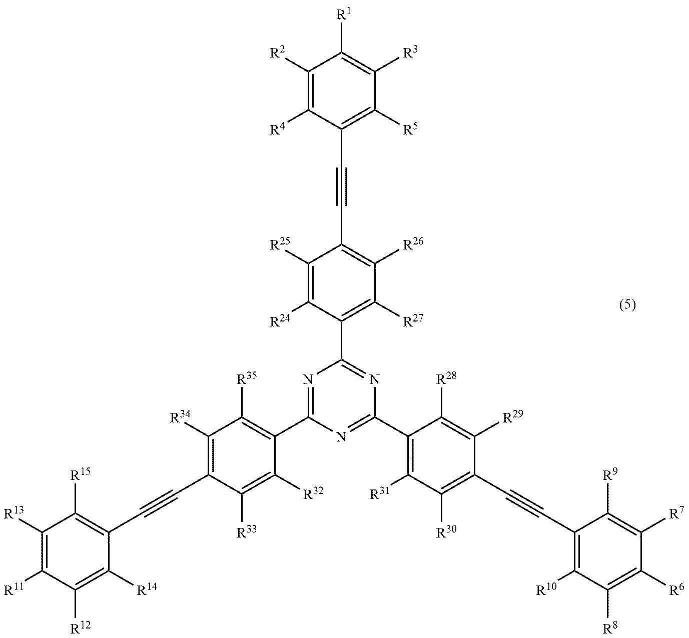

where R²⁴ to R³⁵ each independently include at least one atom selected from the group consisting of H, C, N, O, F, P, S, C1, I, and Br.

In a third aspect of the present disclosure, the non-linear optical material according to the first or second aspect may be one in which, for example, R¹ to R¹⁵, are each independently a hydrogen atom, a halogen atom, an alkyl group, a halogenated alkyl group, an unsaturated hydrocarbon group, a hydroxyl group, a carboxyl group, an alkoxycarbonyl group, an acyl group, an amide group, a nitrile group, an alkoxy group, an acyloxy group, a thiol group, an alkylthio group, a sulfonic acid group, an acylthio group, an alkylsulfonyl group, a sulfonamide group, a primary amino group, a secondary amino group, a tertiary amino group, or a nitro group.

With regard to the second or third aspect, the non-linear optical material has two-photon absorption properties in which high non-linearity is exhibited with respect to light having a wavelength in a short wavelength range.

In a fourth aspect of the present disclosure, the non-linear optical material according to any one of the first to third aspects may be one in which, for example, at least one selected from the group consisting of R¹ to R³, R⁶ to R⁸, and R¹¹ to R¹³ is an electron-donating group or an electron-withdrawing group.

In a fifth aspect of the present disclosure, the non-linear optical material according to any one of the first to fourth aspects may be one in which, for example, at least one selected from the group consisting of R¹ to R³, R⁶ to R⁸, and R⁶ to R¹³ is an alkoxycarbonyl group.

In a sixth aspect of the present disclosure, the non-linear optical material according to any one of the first to fifth aspects may be one in which, for example, at least one selected from the group consisting of R¹ to R³, R⁶ to R⁸, and R¹¹ to R¹³ is —COOC₄H₉ or —COOC₈H₁₇.

With regard to the fourth to sixth aspects, the non-linear optical material has enhanced two-photon absorption properties with respect to light having a wavelength in a short wavelength range.

According to a seventh aspect of the present disclosure, a compound is a compound that is used in a device that utilizes light having a wavelength of greater than or equal to 390 nm and less than or equal to 420 nm, and the compound is represented by formula (1) below,

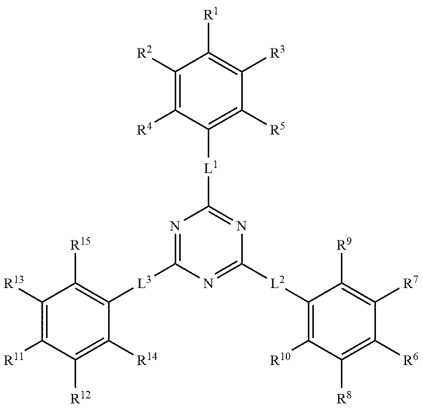

where R¹ to R¹⁵ each independently include at least one atom selected from the group consisting of H, C, N, O, F, P, S, C1, I, and Br, and L¹ to L³ are each independently represented by formula (2) or (3) below,

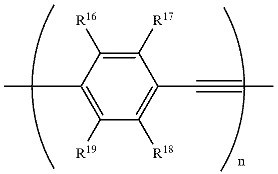

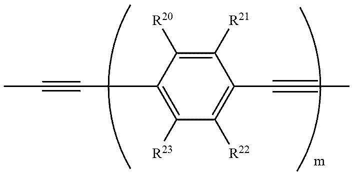

where
R¹⁶ to R¹⁹ each independently include at least one atom selected from the group consisting of H, C, N, O, F, P, S, C1, I, and Br, and n is an integer of 1 to 3, and
R²⁰ to R²³ each independently include at least one atom selected from the group consisting of H, C, N, O, F, P, S, C1, I, and Br, and m is an integer of 1 to 3.

With regard to the seventh aspect, the compound has excellent two-photon absorption properties and low single-photon absorption properties, with respect to light having a wavelength in a short wavelength range. That is, the compound has two-photon absorption properties in which high non-linearity is exhibited with respect to light having a wavelength in a short wavelength range.

According to an eighth aspect of the present disclosure, a recording medium includes a non-linear optical material represented by formula (1) below,

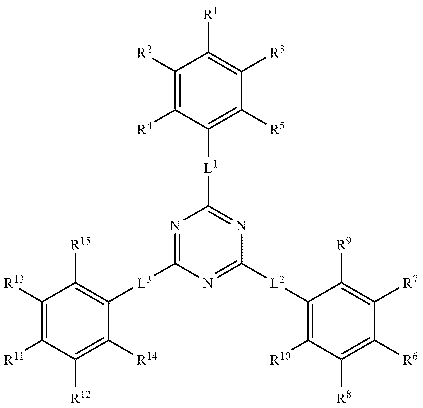

where R¹ to R¹⁵ each independently include at least one atom selected from the group consisting of H, C, N, O, F, P, S, C1, 1, and Br, and L¹ to L¹ are each independently represented by formula (2) or (3) below.

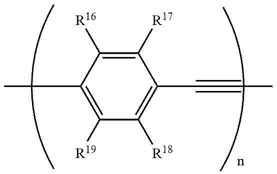

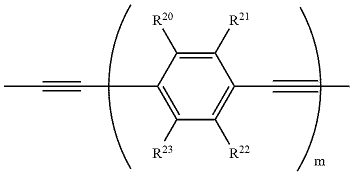

where
R¹⁶ to R¹⁹ each independently include at least one atom selected from the group consisting of H, C, N, O, F, P, S, C1, I, and Br, and n is an integer of 1 to 3, and
R²⁰ to R²³ each independently include at least one atom selected from the group consisting of H, C, N, O, F, P, S, C1, I, and Br, and m is an integer of 1 to 3.

With regard to the eighth aspect, the non-linear optical material has excellent two-photon absorption properties and low single-photon absorption properties, with respect to light having a wavelength in a short wavelength range. That is, the non-linear optical material has two-photon absorption properties in which high non-linearity is exhibited with respect to light having a wavelength in a short wavelength range. Since the recording medium includes the non-linear optical material, the recording medium can record information at a high recording density.

According to a ninth aspect of the present disclosure, a method for recording information includes
providing a light source that emits light having a wavelength of greater than or equal to 390 nm and less than or equal to 420 nm; and
focusing the light from the light source and projecting the light onto a recording region of a recording medium that includes a compound represented by formula (1) below,

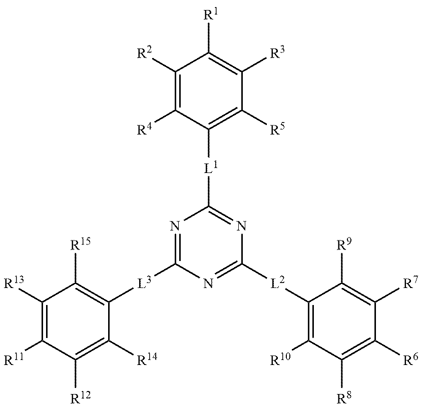

where R¹ to R¹⁵ each independently include at least one atom selected from the group consisting of H, C, N, O, F, P, S, C1, I, and Br, and L¹ to L³ are each independently represented by formula (2) or (3) below,

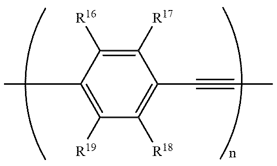

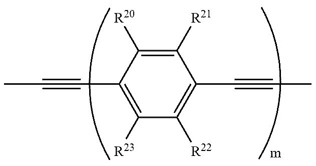

where
R¹⁶ to R¹⁹ each independently include at least one atom selected from the group consisting of H, C, N, O, F, P, S, C1, I, and Br, and n is an integer of 1 to 3, and
R²⁰ to R²³ each independently include at least one atom selected from the group consisting of H, C, N, O, F, P, S, Cl, I, and Br, and m is an integer of 1 to 3.

With regard to the ninth aspect, the compound has excellent two-photon absorption properties and low single-photon absorption properties, with respect to light having a wavelength in a short wavelength range That is, the compound has two-photon absorption properties in which high non-linearity is exhibited with respect to light having a wavelength in a short wavelength range. Since the method for recording information uses a recording medium that includes the compound, the method can record information at a high recording density.

According to a tenth aspect of the present disclosure, a method for reading information is, for example, a method for reading information recorded by the method according to the ninth aspect, and the method for reading information includes
measuring an optical property of the recording region by projecting light onto the recording region of the recording medium; and
determining, based on the optical property, whether there is information recorded in the recording region.

In an eleventh aspect of the present disclosure, the method for reading information according to the tenth aspect may be one in which, for example, the optical property is an intensity of light that reflects off the recording region.

With regard to the tenth or eleventh aspect, identification of recording regions in which information has been recorded can be easily achieved.

Embodiments of the present disclosure will now be described with reference to the drawings. The present disclosure is not limited to the embodiments described below.

A compound A, according to the present embodiment, is represented by formula (1) below.

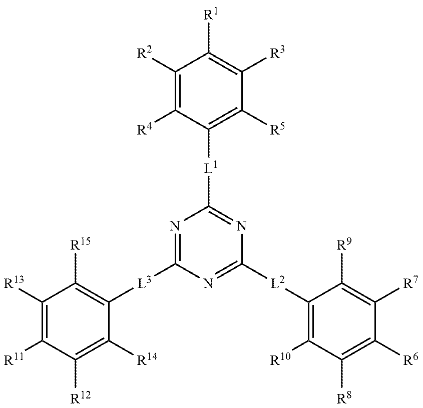

In formula (1), R¹ to R¹⁵ each independently include at least one atom selected from the group consisting of H, C, N, O, F, P, S, C1, I, and Br. R¹to R¹⁵ may each be independently a hydrogen atom, a halogen atom, an alkyl group, a halogenated alkyl group, an unsaturated hydrocarbon group, a hydroxyl group, a carboxyl group, an alkoxycarbonyl group, an acyl group, an amide group, a nitrile group, an alkoxy group, an acyloxy group, a thiol group, an alkylthio group, a sulfonic acid group, an acylthio group, an alkylsulfonyl group, a sulfonamide group, a primary amino group, a secondary amino group, a tertiary amino group, or a nitro group. R¹ to R¹⁵ may each be independently a hydrogen atom, a halogen atom, an alkyl group, a halogenated alkyl group, an unsaturated hydrocarbon group, a hydroxyl group, an alkoxycarbonyl group, an acyl group, an amide group, a nitrile group, an acyloxy group, a thiol group, an alkylthio group, a sulfonic acid group, an acylthio group, an alkylsulfonyl group, a sulfonamide group, a primary amino group, a secondary amino group, a tertiary amino group, or a nitro group. R¹ to R¹⁵ may each be independently a hydrogen atom, a halogen atom, a halogenated alkyl group, an unsaturated hydrocarbon group, a hydroxyl group, an alkoxycarbonyl group, an acyl group, an amide group, a nitrile group, an acyloxy group, a thiol group, a sulfonic acid group, an acylthio group, an alkylsulfonyl group, a sulfonamide group, a primary amino group, a secondary amino group, a tertiary amino group, or a nitro group. R¹ to R¹⁵ may each be independently a hydrogen atom (provided that R¹ to R¹⁵ are not all hydrogen atoms), a halogen atom, a halogenated alkyl group, a hydroxyl group, an alkoxycarbonyl group, an acyl group, an amide group, a nitrile group, an acyloxy group, a thiol group, a sulfonic acid group, an acylthio group, an alkylsulfonyl group, a sulfonamide group, a primary amino group, a secondary amino group, a tertiary amino group, or a nitro group.

Examples of the halogen atom include F, C1, Br, and I. In the present specification, a halogen atom may be referred to as a halogen group.

The number of carbon atoms in the alkyl group is not particularly limited and may be, for example, greater than or equal to 1 and less than or equal to 20. The number of carbon atoms in the alkyl group may be greater than or equal to 1 and less than or equal to 10 or greater than or equal to 1 and less than or equal to 5, so that the synthesis of the compound A can be readily carried out. A solubility of the compound A in a solvent or a resin composition can be adjusted by adjusting the number of carbon atoms in the alkyl group. The alkyl group may be linear, branched, or cyclic. At least one hydrogen atom of the alkyl group may be replaced with a group containing at least one atom selected from the group consisting of N, O, P, and S. Examples of the alkyl group include methyl groups, ethyl groups, propyl groups, butyl groups, a 2-methylbutyl group, pentyl groups, hexyl groups, a 2,3-dimethylhexyl group, heptyl groups, octyl groups, nonyl groups, decyl groups, undecyl groups, dodecyl groups, tridecyl groups, tetradecyl groups, pentadecyl groups, hexadecyl groups, heptadecyl groups, octadecyl groups, nonadecyl groups, eicosyl groups, a 2-methoxybutyl group, and a 6-methoxyhexyl group.

The halogenated alkyl group is a group in which at least one hydrogen atom of an alkyl group is replaced with a halogen atom. The halogenated alkyl group may be a group in which all of the hydrogen atoms of an alkyl group are replaced with a halogen atom. Examples of the alkyl group include alkyl groups mentioned above. Specific examples of the halogenated alkyl group include —CF₃.

The unsaturated hydrocarbon group contains an unsaturated bond, such as a carbon-to-carbon double bond or a carbon-to-carbon triple bond. The number of unsaturated bonds present in the unsaturated hydrocarbon group is, for example, greater than or equal to 1 and less than or equal to 5. The number of carbon atoms in the unsaturated hydrocarbon group is not particularly limited and may be, for example, greater than or equal to 2 and less than or equal to 20, greater than or equal to 2 and less than or equal to 10, or greater than or equal to 2 and less than or equal to 5. The unsaturated hydrocarbon group may be linear, branched, or cyclic. At least one hydrogen atom of the unsaturated hydrocarbon group may be replaced with a group containing at least one atom selected from the group consisting of N, O, P, and S Examples of the unsaturated hydrocarbon group include vinyl groups and ethynyl groups.

The hydroxyl group is represented by —OH. The carboxyl group is represented by —COOH. The alkoxycarbonyl group is represented by —COORₐ. The acyl group is represented by —COR_{b}. The amide group is represented by —CONR_{c}R_{d} The nitrile group is represented by —CN. The alkoxy group is represented by —ORₑ. The acyloxy group is represented by —OCORr. The thiol group is represented by —SH. The alkylthio group is represented by —SR_{g}. The sulfonic acid group is represented by —SO₃H. The acylthio group is represented by —SCORₕ. The alkylsulfonyl group is represented by —SO₂Rᵢ. The sulfonamide group is represented by —SO₂NRⱼRₖ. The primary amino group is represented by —NH₂. The secondary amino group is represented by —NHR₁. The tertiary amino group is represented by —NRₘRₙ. The nitro group is represented by —NO₂. Rₐ to Rₙ are each independently an alkyl group. Examples of the alkyl group include alkyl groups mentioned above. Note that R_{c} and R_{d} in the amide group and Rⱼ and Rₖ in the sulfonamide group may each be independently a hydrogen atom.

Specific examples of the alkoxycarbonyl group include —COOCH₃, —COO(CH2)₃CH₃, and —COO(CH₂)₇CH₃. Specific examples of the acyl group include —COCH₃. Specific examples of the amide group include —CONH₂. Specific examples of the alkoxy group include methoxy groups, ethoxy groups, 2-methoxyethoxy groups, butoxy groups, 2-methylbutoxy groups, 2-methylbutoxy groups, 4-ethylthiobutoxy groups, pentyloxy groups, hexyloxy groups, heptyloxy groups, octyloxy groups, nonyloxy groups, decyloxy groups, undecyloxy groups, dodecyloxy groups, tridecyloxy groups, tetradecyloxy groups, pentadecyloxy groups, hexadecyloxy groups, heptadecyloxy groups, octadecyloxy groups, nonadecyloxy groups, and eicosyloxy groups. Specific examples of the acyloxy group include —OCOCH₃. Specific examples of the acylthio group include SCOCH_{3.} Specific examples of the alkylsulfonyl group include —SO₂CH₃. Specific examples of the sulfonamide group include —SO₂NH₂. Specific examples of the tertiary amino group include —N(CH₃)₂.

At least one selected from the group consisting of R¹ to R³ R⁶ to R⁸, and R¹¹ to R¹³ may be an electron-donating group or an electron-withdrawing group. Regarding R¹ to R³, R⁶ to R⁸, and R¹¹ to R¹³, the greater the electron-donating ability or the electron-withdrawing ability, the more unevenly the electrons are distributed in the compound A. In instances where the electrons in the compound A are highly unevenly distributed, the electrons tend to move considerably in the compound A when the compound A is excited. In this case, the compound A tends to have enhanced two-photon absorption properties. In other words, when at least one selected from the group consisting of R¹ to R³, R⁶ to R⁸, and R¹¹ to R¹³ is an electron-donating group or an electron-withdrawing group, the compound A tends to have a large two-photon absorption cross section.

The electron-withdrawing group is a substituent that, for example, has a positive σₚ value, where the σₚ value is the substituent constant in the Hammett equation. Examples of the electron-withdrawing group include halogen atoms, carboxyl groups, nitro groups, thiol groups, sulfonic acid groups, acyloxy groups, alkylthio groups, alkylsulfonyl groups, sulfonamide groups, acyl groups, acylthio groups, alkoxycarbonyl groups, and halogenated alkyl groups. At least one selected from the group consisting of R¹ to R³, R⁶ to R⁸, and R¹¹ to R¹³ may be an alkoxycarbonyl group and may be —COOC₄H₉ or —COOC₈H ₁₇.

The electron-donating group is a substituent that, for example, has a negative σₚ value, where the σₚ value is as described above. Examples of the electron-donating group include alkyl groups, alkoxy groups, hydroxyl groups, and amino groups.

R⁴, R⁵, R⁹, R¹⁰, R¹⁴, and R¹⁵ may each have a small volume. In this case, steric hindrance is unlikely to occur in R⁴, R⁵, R⁹, R¹⁰, R¹⁴, and R¹⁵. Accordingly, planarity of the π electron conjugated system tends to be improved in the compound A. When the π electron conjugated system of the compound A has high planarity, the compound A tends to have a large two-photon absorption cross section. R⁴, R⁵, R⁹, R¹⁰, R¹⁴, and R¹⁵ may each be a hydrogen atom.

In formula (1), L¹ to L³ are each independently represented by formula (2) or (3) below.

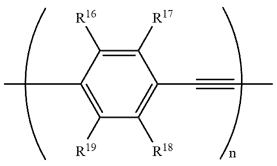

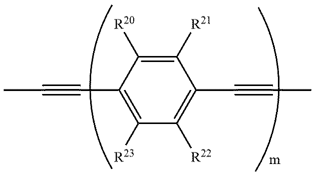

In formula (2), R¹⁶ to R¹⁹ each independently include at least one atom selected from the group consisting of H, C, N, O, F, P, S, C1, I, and Br. R¹⁶ to R¹⁹ may each be independently a hydrogen atom or any of the substituents mentioned above for R¹ to R¹⁵. R¹⁶ to R¹⁹ may each have a small volume. In this case, steric hindrance is unlikely to occur in R¹⁶ to R¹⁹ Accordingly, the planarity of the π electron conjugated system tends to be improved in the compound A, and, consequently, the compound A tends to have a large two-photon absorption cross section. R¹⁶ to R¹⁹ may each be a hydrogen atom. In formula (2), n is an integer of 1 to 3. The greater the value of n, the more the π electron conjugated system is extended, which results in a tendency for the compound A to have an increased two-photon absorption cross section. n may be 1 when a solubility of the compound A is taken into account.

In formula (3), R²⁰ to R²³ each independently include at least one atom selected from the group consisting of H, C, N, O, F, P, S, C1, I, and Br. R²⁰ to R²³ may each be independently a hydrogen atom or any of the substituents mentioned above for R¹ to R¹⁵. R²⁰ to R²³ may each have a small volume. In this case, steric hindrance is unlikely to occur in R²⁰ to R²³. Accordingly, the planarity of the π electron conjugated system tends to be improved in the compound A, and, consequently, the compound A tends to have a large two-photon absorption cross section. R²⁰ to R²³ may each be a hydrogen atom. In formula (3), m is an integer of 1 to 3. The greater the value of m, the more the π electron conjugated system is extended, which results in a tendency for the compound A to have an increased two-photon absorption cross section. m may be 1 when the solubility of the compound A is taken into account.

L¹ to L³ may be identical to or different from one another. For example, L¹ to L³ may each be represented by formula (2). For example, the compound A is a compound B, which is represented by formula (5) below.

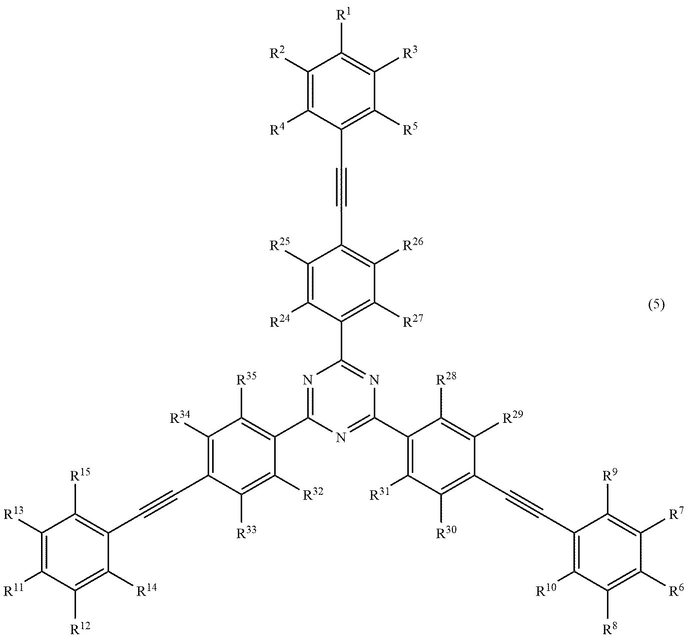

In formula (5), R²⁴ to R³⁵ each independently include at least one atom selected from the group consisting of H, C, N, O, F, P, S, C1, I, and Br. R²⁴ to R³⁵ each correspond to a corresponding one of R¹⁶ to R¹⁹, described above.

In instances where the compound A is a compound C, which is represented by formula (4) below, at least one selected from the group consisting of R¹, R⁶, and R¹¹ is a halogen atom, a halogenated alkyl group, an unsaturated hydrocarbon group, a hydroxyl group, an alkoxycarbonyl group, an acyl group, an amide group, an acyloxy group, a thiol group, an alkylthio group, a sulfonic acid group, an acylthio group, an alkylsulfonyl group, a sulfonamide group, a primary amino group, a secondary amino group, or a nitro group. In this instance, R¹, R⁶, and R¹¹ may each be independently a hydrogen atom, a halogen atom, a halogenated alkyl group, an unsaturated hydrocarbon group, a hydroxyl group, an alkoxycarbonyl group, an acyl group, an amide group, a nitrile group, an acyloxy group, a thiol group, a sulfonic acid group, an acylthio group, an alkylsulfonyl group, a sulfonamide group, a primary amino group, a secondary amino group, a tertiary amino group, or a nitro group. R¹, R⁶, and R¹¹ may each be independently a hydrogen atom (provided that R¹, R⁶, and R¹¹ are not all hydrogen atoms), a halogen atom, a halogenated alkyl group, a hydroxyl group, an alkoxycarbonyl group, an acyl group, an amide group, a nitrile group, an acyloxy group, a thiol group, a sulfonic acid group, an acylthio group, an alkylsulfonyl group, a sulfonamide group, a primary amino group, a secondary amino group, a tertiary amino group, or a nitro group. In some instances, R¹, R⁶, and R¹¹ in formula (4) may each be a substituent other than the substituents mentioned above.

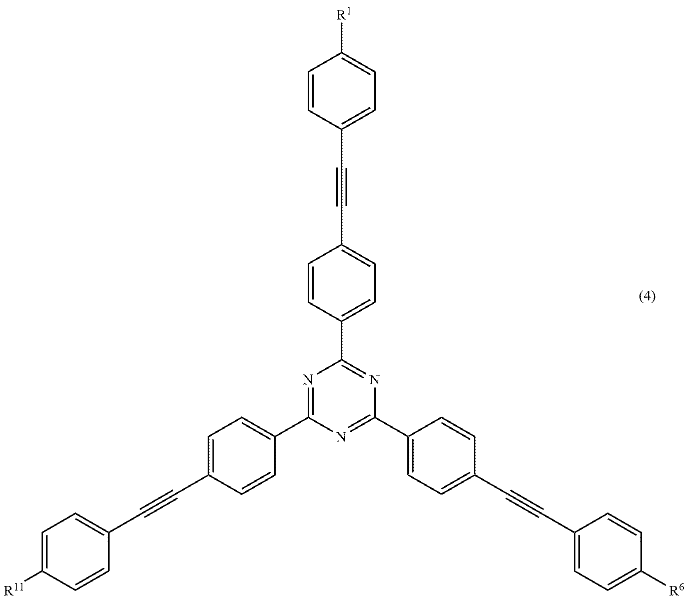

Specific examples of the compound B, which is represented by formula (5), include a compound D, which is represented by formula (6) below, and a compound E, which is represented by formula (7) below.

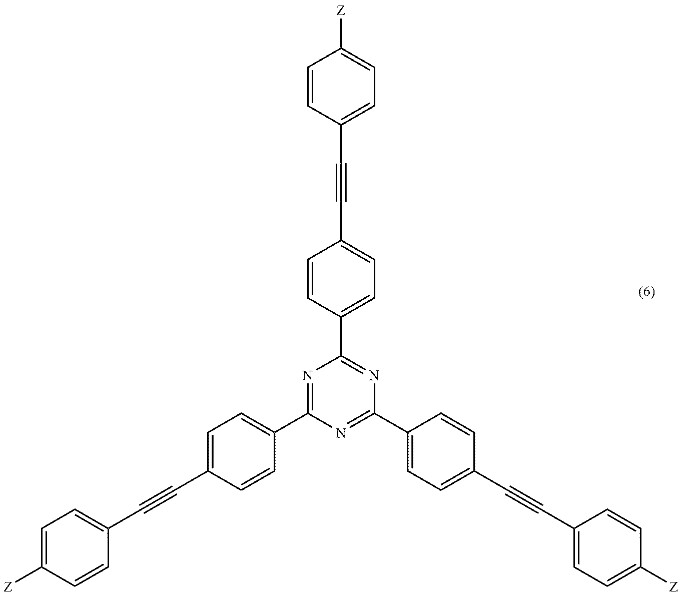

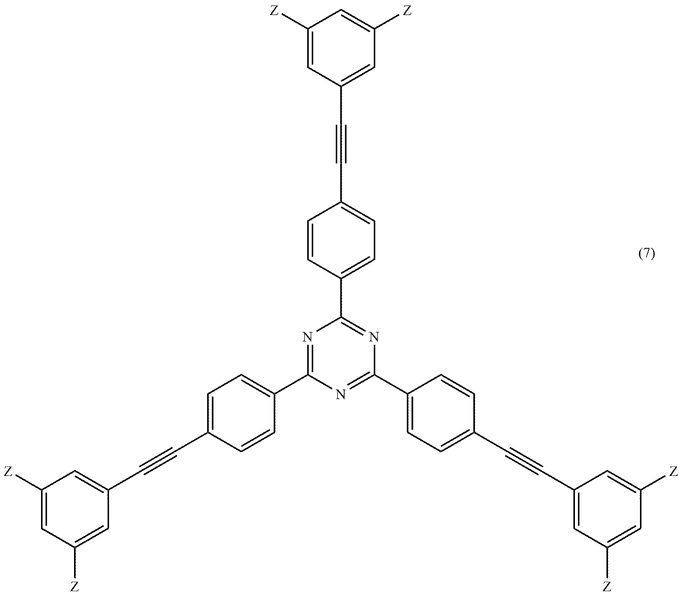

In formula (6), Z’s are identical to one another. Z’s correspond to respective ones of R¹, R⁶, and R¹¹ of formula (5) Specific examples of Z are shown in Table 1 below. In formula (6), Z’s may be —COOC₄H₉ or —COOC₈H₁₇. In some instances, Z’s in formula (6) may be —COOH.

TABLE 1

| | Z |
|---|---|
| 1 | —H |
| 2 | — F |
| 3 | —CH₃ |
| 4 | —C₂H₅ |
| 5 | —CF₃ |
| 6 | —OH |
| 7 | —COOH |
| 8 | —COOCH₃ |
| 9 | —COOC₄H₉ |
| 10 | —COOC₈H₁₇ |
| 11 | —COCH₃ |
| 12 | —CONH₂ |
| 13 | — CN |
| 14 | —OCH₃ |
| 15 | —OCOCH₃ |
| 16 | —SH |
| 17 | —SO₃H |
| 18 | — SCOCH₃ |
| 19 | — SO₂CH₃ |
| 20 | — SO₂NH₂ |
| 21 | —NH₂ |
| 22 | N (CH₃) 2 |
| 23 | —NO₂ |
| 24 | —C (CH₃) ₃ |

In formula (7), Z’s are identical to one another. Z’s correspond to respective ones of R², R³, R⁷, R⁸, R¹², and R¹³ of formula (5). Z’s may be a hydrogen atom or a substituent as shown in Table 1. In formula (7), Z’s may be —COOC₄H₉ or —COOC₈H₁₇. In some instances, Z’s in formula (7) may be —COOH.

L¹ to L³ of formula (1) may each be represented by formula (3). For example, the compound A may be a compound F, which is represented by formula (8) below.

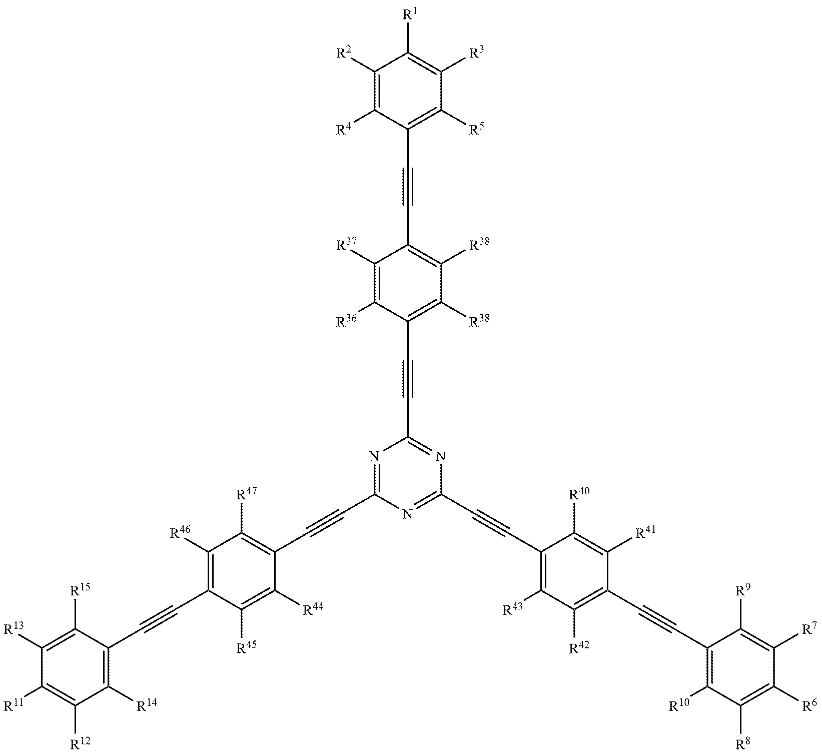

In formula (8), R³⁶ to R⁴⁷ each independently include at least one atom selected from the group consisting of H, C, N, O, F, P, S, Cl, I, and Br. R³⁶ to R⁴⁷ each correspond to a corresponding one of R²⁰ to R²³, described above.

Specific examples of the compound F include a compound G, which is represented by formula (9) below, and a compound H, which is represented by formula (10) below.

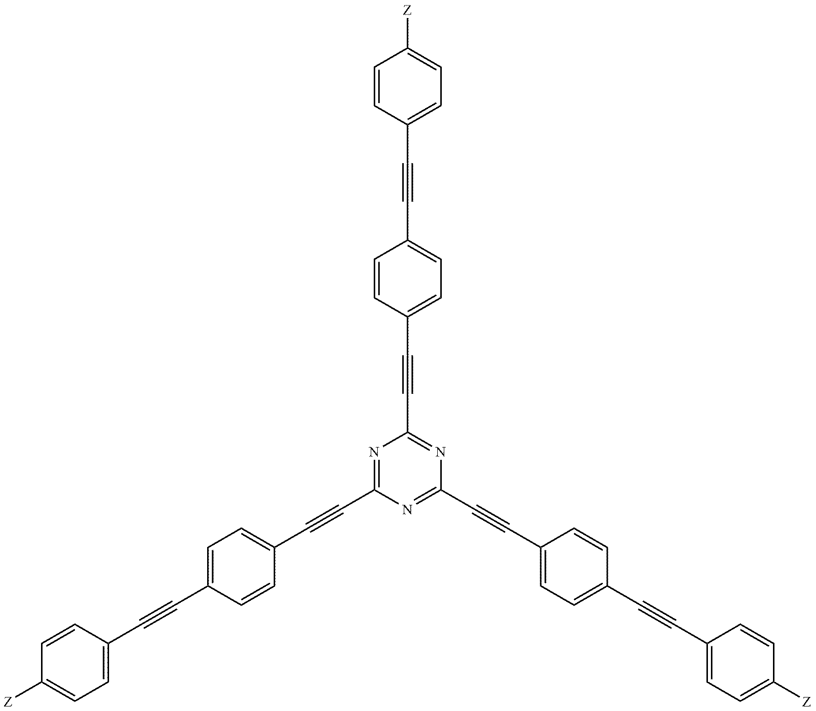

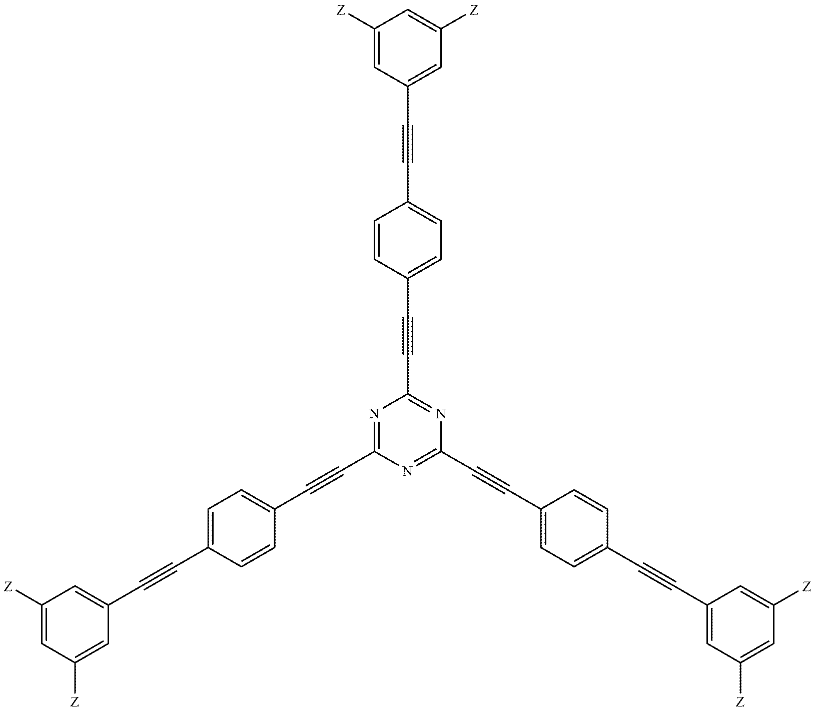

In formula (9), Z’s are identical to one another. Z’s correspond to respective ones of R¹, R⁶, and R¹¹ of formula (8) Z’s may be a hydrogen atom or a substituent as shown in Table 1.

In formula (10), Z’s are identical to one another. Z’s correspond to respective ones of R², R³, R⁷, R⁸, R¹², and R¹³ of formula (8). Z’s may be a hydrogen atom or a substituent as shown in Table 1.

Methods for synthesizing the compound D, which is represented by formula (6), and synthesizing the compound E, which is represented by formula (7), are not particularly limited. The compounds D and E can be synthesized, for example, by using the following method. First, a compound I, which is represented by formula (11) below, is prepared.

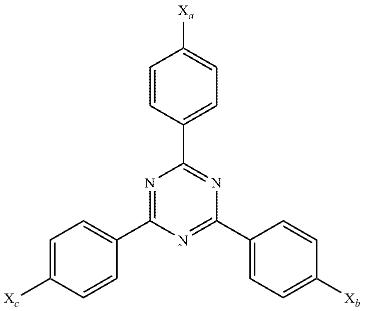

In formula (11), Xₐ to X_{c} are each independently a substituent that is reactive in a coupling reaction. Representative examples of such substituents include halogen groups. Xₐ to X_{c} may be an ethynyl group. Next, a coupling reaction is carried out between the compound I and a compound J, which has an appropriate structure. In this manner, the compound D or E can be synthesized. The structure of the compound J depends on the structure of the target compound. The conditions for the coupling reaction can be appropriately adjusted in accordance with the structures of the compounds I and J, for example.

Methods for synthesizing the compound G, which is represented by formula (9), and synthesizing the compound H, which is represented by formula (10), are not particularly limited. The compounds G and H can be synthesized, for example, by using the following method. First, a compound K, which is represented by formula (12) below, is prepared.

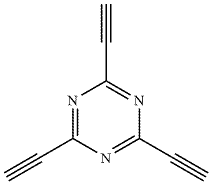

Next, a coupling reaction is carried out between the compound K and a compound L, which has an appropriate structure. In this manner, the compound G or H can be synthesized. The structure of the compound L depends on the structure of the target compound. For example, the compound L contains a substituent that is reactive in a coupling reaction. Representative examples of such substituents include halogen groups. The conditions for the coupling reaction can be appropriately adjusted in accordance with the structures of the compounds K and L, for example.

The compound A, which is represented by formula (1), has excellent two-photon absorption properties and low single-photon absorption properties, with respect to light having a wavelength in a short wavelength range. For example, when light having a wavelength of 405 nm is projected onto the compound A, two-photon absorption occurs while substantially no single-photon absorption occurs, in the compound A.

The two-photon absorption cross section of the compound A with respect to light having a wavelength of 405 nm may be greater than 410 GM, greater than 500 GM, greater than or equal to 1000 GM, greater than or equal to 1500 GM, or greater than or equal to 1700 GM. The upper limit of the two-photon absorption cross section of the compound A is not particularly limited and is, for example, 5000 GM. The two-photon absorption cross section can be measured, for example, by using the Z-scan method described in J. Opt. Soc. Am. B, 2003, Vol. 20, p. 529. The Z-scan method is widely used as a method for measuring a non-linear optical constant. In the Z-scan method, a measurement sample is moved in a region at and near the focal point at which a laser beam converges, along the direction in which the beam is projected In this process, changes in the amount of light that has passed through the measurement sample are recorded. In the Z-scan method, a power density of the incident light varies depending on the position of the measurement sample. Accordingly, in instances where a measurement sample performs non-linear absorption, the amount of light that passes through the measurement sample decreases when the measurement sample is located at or near the focal point of the laser beam. The two-photon absorption cross section can be calculated by performing fitting of the changes in the amount of light that has passed, with respect to a theoretical curve of the amount of light that passes, which is an amount predicted from, for example, an intensity of the incident light, a thickness of the measurement sample, and a concentration of the compound A in the measurement sample.

The two-photon absorption cross section may be a calculated value calculated by computational chemistry. Several methods have been proposed for estimating the two-photon absorption cross section by using computational chemistry. For example, a calculated value of the two-photon absorption cross section can be calculated in accordance with a secondary non-linear response theory described in J. Chem. Theory Comput. 2018, Vol. 14, p. 807.

A molar extinction coefficient of the compound A with respect to light having a wavelength of 405 nm may be less than or equal to 800 L/(mol cm), less than or equal to 500 L/(mol · cm), less than or equal to 210 L/(mol · cm), or less than or equal to 100 L/(mol ·cm). The lower limit of the molar extinction coefficient of the compound A is not particularly limited and is, for example, 0.01 L/(mol ·cm). For example, the molar extinction coefficient can be measured by using a method according to the specifications of Japanese Industrial Standards (JIS) K 0115:2004. In the measurement of the molar extinction coefficient, a light source that projects light having a photon density at which substantially no two-photon absorption occurs in the compound A is to be used. The molar extinction coefficient can be used as an index of single-photon absorption.

The molar extinction coefficient may be a calculated value calculated by a quantum chemistry calculation program. Examples of quantum chemistry calculation programs that can be used include Gaussian 16 (available from Gaussian, Inc.).

In instances where the compound A performs two-photon absorption, the compound A absorbs approximately twice as much energy as the energy of the light projected onto the compound A. Light having energy approximately twice as much as the energy of light having a wavelength of 405 nm has a wavelength of, for example, 200 nm. That is, when light having a wavelength of approximately 200 nm is projected onto the compound A, single-photon absorption may occur in the compound A. In addition, in the compound A, single-photon absorption may occur in association with light having a wavelength near the wavelength range in which two-photon absorption occurs.

A quantum yield of fluorescence in the compound A is not particularly limited and is, for example, greater than or equal to 0% and less than or equal to 50%. The quantum yield of fluorescence may be less than or equal to 30% or less than or equal to 20%. Specifically, in the present specification, the “quantum yield” refers to an internal quantum yield. A wavelength of the fluorescent light emitted by the compound A may be greater than or equal to 405 nm and less than or equal to 660 nm and, in some instances, may be greater than or equal to 350 nm and less than or equal to 650 nm. The quantum yield of fluorescence can be measured, for example, by using a commercially available absolute PL quantum yield spectrometer.

For example, the compound A, which is represented by formula (1), can be used as a component of a light-absorbing material. For example, the light-absorbing material includes the compound A as a major component. The “major component” refers to the most abundant component in the light-absorbing material in terms of a weight ratio. For example, the light-absorbing material consists essentially of the compound A. The phrase “consists essentially of” means that other components that change an intrinsic feature of the mentioned material are excluded. Note that the light-absorbing material may contain impurities in addition to the compound A. For example, the light-absorbing material serves as a multi-photon absorbing material, such as a two-photon absorbing material. In particular, light-absorbing materials including the compound A have two-photon absorption properties in which high non-linearity is exhibited with respect to light having a wavelength in a short wavelength range.

That is, according to another aspect, the present disclosure provides a light-absorbing material including a compound represented by formula (1) below.

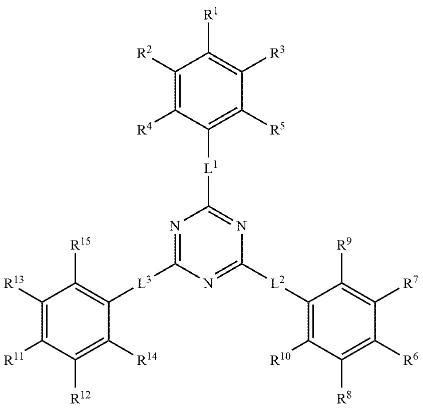

In formula (1), R¹ to R¹⁵ each independently include at least one atom selected from the group consisting of H, C, N, O, F, P, S, Cl, I, and Br, and L¹ to L³ are each independently represented by formula (2) or (3) below.

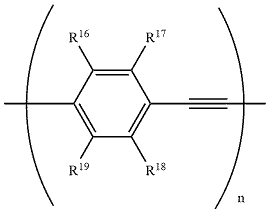

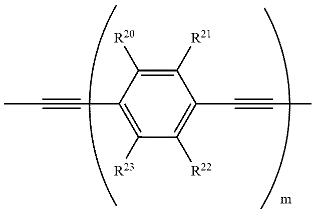

In formula (2), R¹⁶ to R¹⁹ each independently include at least one atom selected from the group consisting of H, C, N, O, F, P, S, Cl, I, and Br, and n is an integer of 1 to 3 In formula (3), R²⁰ to R²³ each independently include at least one atom selected from the group consisting of H, C, N, O, F, P, S, Cl, I, and Br, and m is an integer of 1 to 3.

In instances where the compound described above is represented by formula (4) below, at least one selected from the group consisting of R¹, R⁶, and R¹¹ is a halogen atom, a halogenated alkyl group, an unsaturated hydrocarbon group, a hydroxyl group, a carboxyl group, an alkoxycarbonyl group, an acyl group, an amide group, an acyloxy group, a thiol group, an alkylthio group, a sulfonic acid group, an acylthio group, an alkylsulfonyl group, a sulfonamide group, a primary amino group, a secondary amino group, or a nitro group.

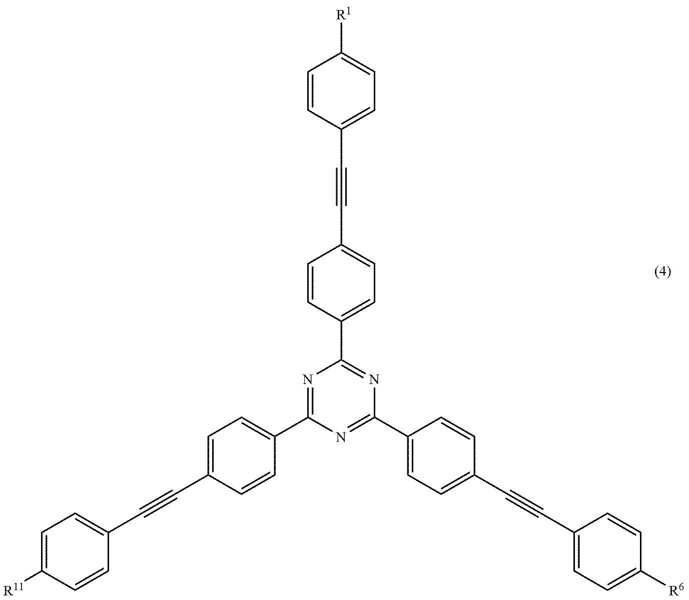

For example, the compound A is used in devices that utilize light having a wavelength in a short wavelength range. Examples of such devices include recording media, additive manufacturing apparatuses, and fluorescence microscopes. Examples of the recording media include three-dimensional optical memories. Specific examples of the three-dimensional optical memories include three-dimensional optical discs. Examples of the additive manufacturing apparatuses include stereolithography apparatuses, such as 3D printers. Examples of the fluorescence microscopes include two-photon fluorescence microscopes. Light used in these devices has a high photon density at or near the focal point, for example. A power density of the light used in these devices, at or near the focal point, is, for example, greater than or equal to 0.1 W/cm2 and less than or equal to 1.0×10²⁰ W/cm². The power density of the light at or near the focal point may be greater than or equal to 1.0 W/cm², greater than or equal to 1.0×10² W/cm², or greater than or equal to 1.0×10⁵ W/cm². Examples of light sources that can be used in the devices include femtosecond lasers, such as titanium-sapphire lasers, and pulsed lasers with a pulse width in a picosecond to nanosecond range, such as semiconductor lasers

That is, according to another aspect, the present disclosure provides a compound that is used in devices that utilize light having a wavelength of greater than or equal to 390 nm and less than or equal to 420 nm and is represented by formula (1) below.

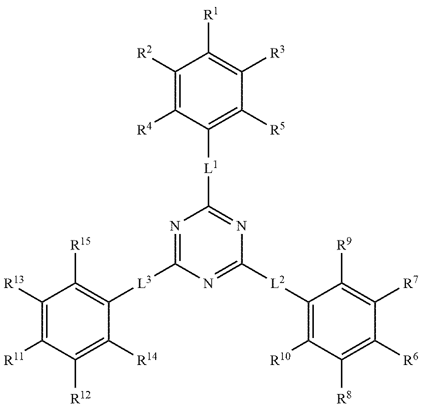

In formula (1), R¹ to R¹⁵ each independently include at least one atom selected from the group consisting of H, C, N, O, F, P, S, Cl, I, and Br, and L¹ to L³ are each independently represented by formula (2) or (3) below.

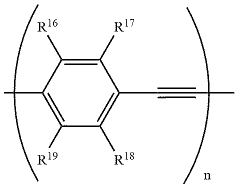

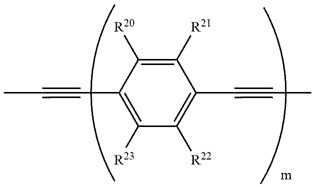

In formula (2), R¹⁶ to R¹⁹ each independently include at least one atom selected from the group consisting of H, C, N, O, F, P, S, Cl, I, and Br, and n is an integer of 1 to 3. In formula (3), R²⁰ to R²³ each independently include at least one atom selected from the group consisting of H, C, N, O, F, P, S, Cl, I, and Br, and m is an integer of 1 to 3.

Recording media include, for example, a thin film referred to as a recording layer In the recording media, information is recorded in the recording layer. For example, the thin film serving as the recording layer includes the compound A.

That is, according to still another aspect, the present disclosure provides a recording medium including a non-linear optical material represented by formula (1) below.

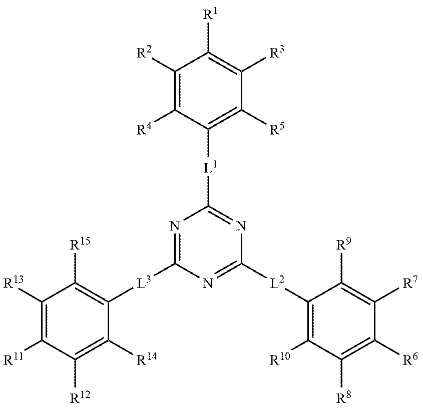

In formula (1), R¹ to R¹⁵ each independently include at least one atom selected from the group consisting of H, C, N, O, F, P, S, Cl, I, and Br, and L¹ to L³ are each independently represented by formula (2) or (3) below.

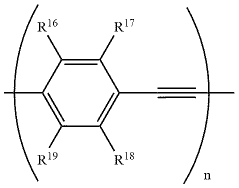

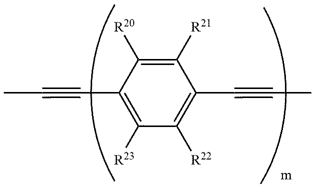

In formula (2), R¹⁶ to R¹⁹ each independently include at least one atom selected from the group consisting of H, C, N, O, F, P, S, Cl, I, and Br, and n is an integer of 1 to 3. In formula (3), R²⁰ to R²³ each independently include at least one atom selected from the group consisting of H, C, N, O, F, P, S, Cl, I, and Br, and m is an integer of 1 to 3.

The recording layer may further include a polymeric compound that serves as a binder, in addition to the compound A. The recording medium may include a dielectric layer in addition to the recording layer. For example, the recording medium includes multiple recording layers and multiple dielectric layers. In the recording medium, the recording layers and the dielectric layers may be alternately layered.

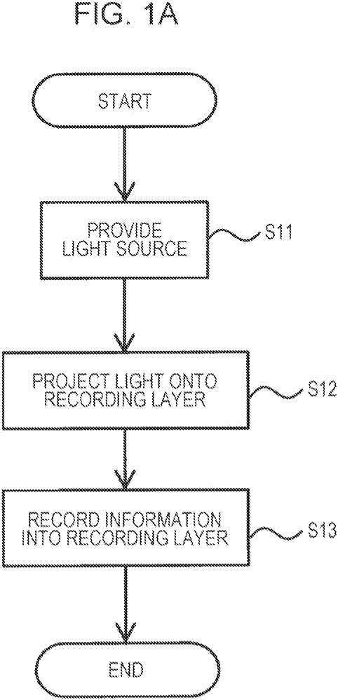

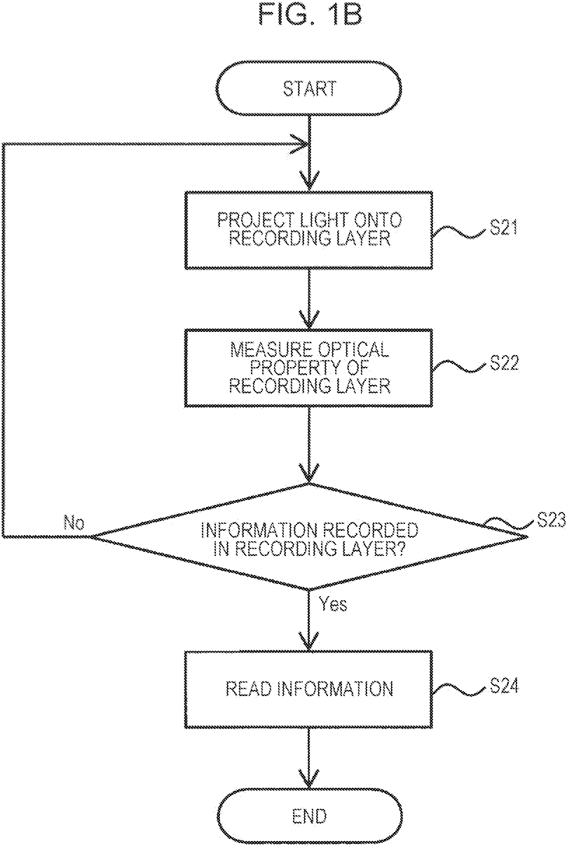

Now, a method for recording information will be described; the method uses the recording medium described above. FIG. 1A is a flowchart illustrating the method for recording information, which is a method using the recording medium described above. First, in step S11, a light source that emits light having a wavelength of greater than or equal to 390 nm and less than or equal to 420 nm is provided. Examples of the light source that can be used include femtosecond lasers, such as titanium-sapphire lasers. Other examples of the light source that can be used include pulsed lasers with a pulse width in a picosecond to nanosecond range, such as semiconductor lasers. Next, in step S12, the light from the light source is focused by using a lens or the like and projected onto a recording layer of the recording medium. Specifically, the light from the light source is focused by using a lens or the like and projected onto a recording region of the recording medium. A power density of the light at or near the focal point is, for example, greater than or equal to 0.1 W/cm² and less than or equal to 1.0× 10²⁰ W/cm². The power density of the light at or near the focal point may be greater than or equal to 1.0 W/cm², greater than or equal to 1.0×10² W/cm², or greater than or equal to 1.0× 10⁵ W/cm². In the present specification, the “recording region” refers to a spot that exists in the recording layer and at which information can be recorded when light is projected onto the spot.

In the recording region, as a result of the projection of light, a physical change or a chemical change occurs. For example, when the compound A has absorbed light and then returns from the transition state to the ground state, heat is generated. The heat denatures the binder that exists in the recording region. As a result, optical properties of the recording region change. For example, changes occur in an intensity of light that reflects off the recording region, a reflectance of light in the recording region, an absorptance of light in the recording region, a refractive index of light in the recording region, and the like. In the recording region, as a result of the projection of light, changes may also occur in an intensity of the fluorescent light that is emitted from the recording region or a wavelength of the fluorescent light that is emitted from the recording region. Accordingly, information can be recorded into the recording layer, specifically, into the recording region (step S13).

That is, according to still another aspect, the present disclosure provides a method for recording information, and the method includes
providing a light source that emits light having a wavelength of greater than or equal to 390 nm and less than or equal to 420 nm; and
focusing the light from the light source and projecting the light onto a recording region of a recording medium that includes a compound represented by formula (1) below.

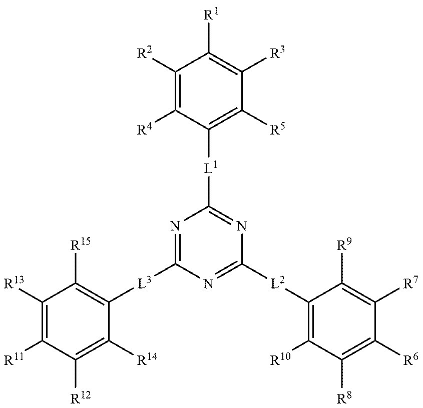

In formula (1), R¹ to R¹⁵ each independently include at least one atom selected from the group consisting of H, C, N, O, F, P, S, Cl, I, and Br, and L¹ to L³ are each independently represented by formula (2) or (3) below.

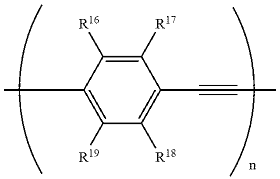

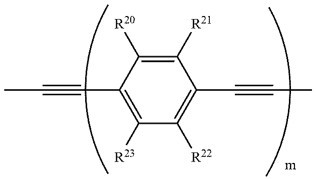

In formula (2), R¹⁶ to R¹⁹ each independently include at least one atom selected from the group consisting of H, C, N, O, F, P, S, Cl, I, and Br, and n is an integer of 1 to 3. In formula (3), R²⁰ to R²³ each independently include at least one atom selected from the group consisting of H, C, N, O, F, P, S, Cl, I, and Br, and m is an integer of 1 to 3.

Now, a method for reading information will be described; the method uses the recording medium described above. FIG. 1B is a flowchart illustrating the method for reading information, which is a method using the recording medium described above. First, in step S21, light is projected onto a recording layer of the recording medium. Specifically, light is projected onto a recording region of the recording medium. The light used in step S21 may be identical to or different from the light used to record information into the recording medium. Next, in step S22, one or more optical properties of the recording layer are measured. Specifically, one or more optical properties of the recording region are measured. Examples of the one or more optical properties of the recording region to be measured in step S22 include an intensity of light that reflects off the recording region. Further examples of the one or more optical properties of the recording region to be measured in step S22 include a reflectance of light in the recording region, an absorptance of light in the recording region, a refractive index of light in the recording region, an intensity of the fluorescent light emitted from the recording region, and a wavelength of the fluorescent light emitted from the recording region

Next, in step S23, based on the one or more optical properties of the recording layer, a determination is made as to whether there is information recorded in the recording layer. For example, in an instance where the intensity of light that reflects off the recording region is less than or equal to a specific value, a determination is made that there is information recorded in the recording layer. On the other hand, in an instance where the intensity of light that reflects off the recording region is greater than the specific value, a determination is made that there is no information recorded in the recording layer. In the instance where a determination is made that there is no information recorded in the recording layer, the process returns to step S21, and the same operation is performed on another recording layer. In an instance where a determination is made that there is information recorded in the recording layer, the information is read in step S24.

The method for recording information and the method for reading information, which use the recording medium described above, can be carried out, for example, by using a recording apparatus known in the art. For example, the recording apparatus includes a light source, a meter, and a controller; the light source projects light onto the recording region of the recording medium, the meter measures the optical properties of the recording region, and the controller controls the light source and the meter.

Additive manufacturing apparatuses perform additive manufacturing, for example, by projecting light onto a photocurable resin composition and curing the resin composition. For example, a photocurable resin composition for stereolithography includes the compound A. The photocurable resin composition includes, for example, a polymerizable compound and a polymerization initiator, in addition to the compound A. The photocurable resin composition may further include one or more additives, such as a binder resin. The photocurable resin composition may include an epoxy resin.

Fluorescence microscopes enable examination of fluorescence, which is, for example, fluorescence emitted from a fluorochrome material when light is projected onto a biological sample containing the fluorochrome material. For example, a fluorochrome material to be added to a biological sample includes the compound A.

### Examples

The present disclosure will now be described in more detail with reference to examples. Note that the examples described below are merely illustrative, and the present disclosure is not limited to the examples described below. In the present disclosure, the compounds used in the examples are denoted as “Compound (X)-Y”. “X” denotes the structural formula of the compound. “Y” denotes the type of Z in formula (X). For example, “Compound (6)-7” denotes that the compound is a compound represented by formula (6) and in which Z is substituent 7 (—COOH) as shown in Table 1.

### Synthesis of Compound (6)-7

First, 2,4,6-tris(4-bromophenyl)-1,3,5-triazine and 4-ethynyl-benzoic acid methyl were dissolved in triethylamine. Furthermore, catalytic amounts of triphenylphosphine, bis(triphenylphosphine)palladium(II) dichloride, and copper (I) iodide were added to the resulting solution. Next, the solution was stirred at room temperature for 16 hours. A neutralization process was performed on the resulting reaction solution by adding hydrochloric acid to the resulting reaction solution. Next, an extraction process was performed on the reaction solution by using ethyl acetate. Magnesium sulfate was added to the resulting extracted liquid to dehydrate the extracted liquid. Next, the magnesium sulfate was filtered out from the extracted liquid. The resulting filtrate was concentrated by using a rotary evaporator. The resulting concentrated liquid was purified by using silica gel column chromatography, and, accordingly, a precursor of compound (6)-7 was obtained.

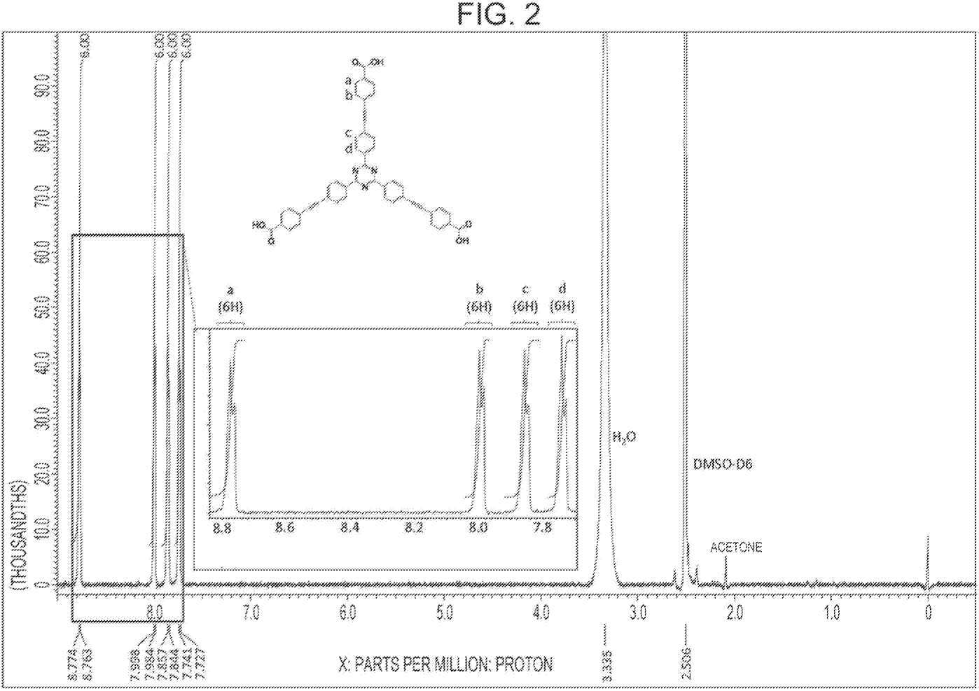

Next, the precursor of compound (6)-7 was dissolved in a liquid mixture containing tetrahydrofuran and methanol (v/v=1:1). An aqueous sodium hydroxide solution was added to the resulting solution, which was then heated at reflux with stirring overnight. After the reaction in the solution was completed, dilute hydrochloric acid was added to the solution. Accordingly, the solution was acidified, and a solid precipitated out. The solid was washed with purified water, and, accordingly, compound (6)-7, which was a white solid, was obtained. Compound (6)-7 was identified by using ¹H-NMR. FIG. 2 is a graph illustrating the ¹H-NMR spectrum of compound (6)-7 The ¹H-NMR spectrum of compound (6)-7 was as follows: ¹H-NMR (600 MHz, DMSO-D6) δ8.77 (d, J=6.9 Hz, 6 H), 7.99 (d, J=8.3 Hz, 6 H), 7.85 (d, J=7.6 Hz, 6 H), 7.73 (d, J=8.3 Hz, 6 H).

### Synthesis of Compound (6)-9

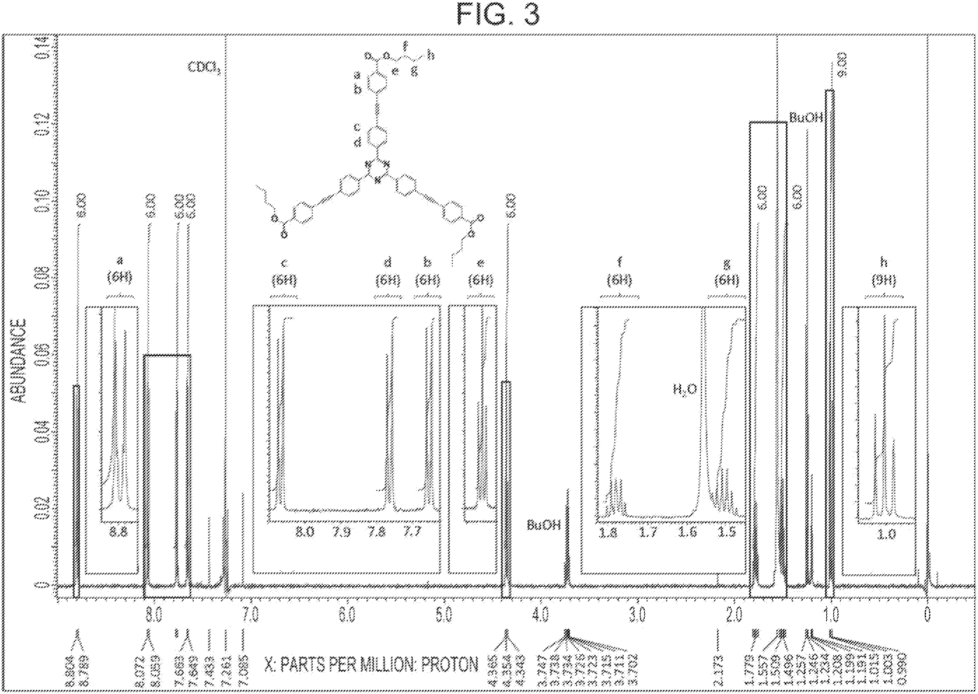

First, compound (6)-7, described above, was added to a butanol solvent to prepare a suspension liquid. Next, thionyl chloride was added to the suspension liquid, which was then heated at reflux with stirring overnight. A white solid was filtered out from the resulting reaction solution and washed with methanol. An extraction process was performed on the resulting solid by using chloroform Magnesium sulfate was added to the resulting extracted liquid to dehydrate the extracted liquid. Next, the magnesium sulfate was filtered out from the extracted liquid. The resulting filtrate was concentrated by using a rotary evaporator. The resulting concentrated liquid was purified by using silica gel column chromatography, and, accordingly, compound (6)-9, which was a white solid, was obtained. Compound (6)-9 was identified by using ¹H-NMR. FIG. 3 is a graph illustrating the ¹H-NMR spectrum of compound (6)-9. The ¹H-NMR spectrum of compound (6)-9 was as follows: ¹H-NMR (600 MHz, CHLOROFORM-D) δ8.80 (d, J=9.0 Hz, 6 H), 8.07 (d, J=8.3 Hz, 6 H), 7.76 (d, J=8.3 Hz, 6 H), 7.66 (d, J=8.3 Hz, 6 H), 4.35 (t, J=6.9 Hz, 6 H), 1.76-1.80 (m, 6 H), 1.47-1.52 (m, 6 H), 1.00 (t, J=7.6 Hz, 9 H)

### Synthesis of Compound (6)-10

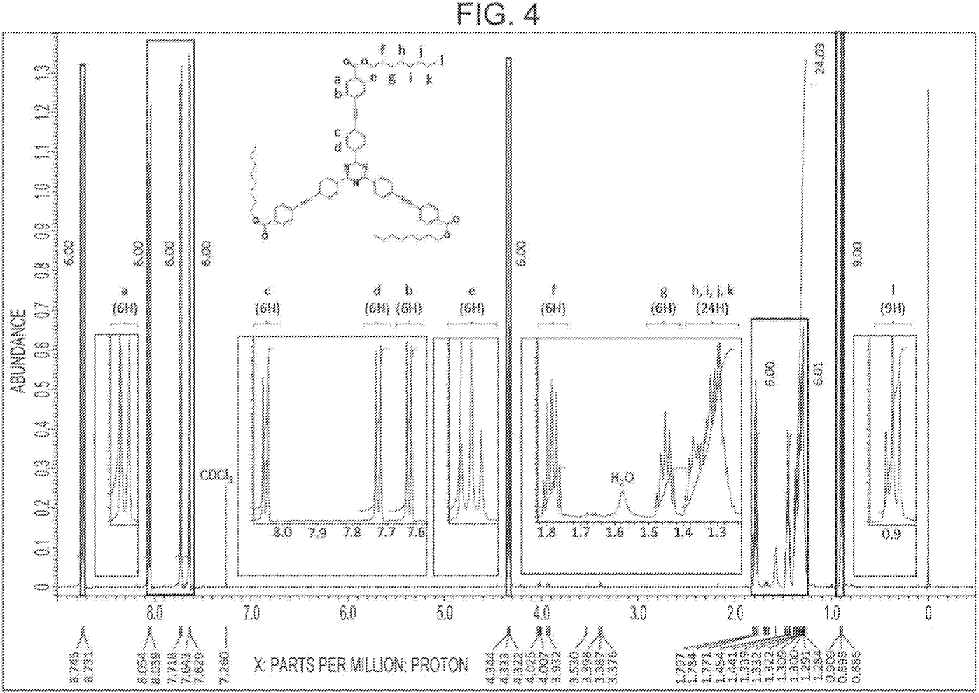

First, compound (6)-7, described above, was added to an octanol solvent to prepare a suspension liquid. Next, thionyl chloride was added to the suspension liquid, which was then heated at reflux with stirring overnight. A white solid was filtered out from the resulting reaction solution and washed with methanol. An extraction process was performed on the resulting solid by using chloroform. Magnesium sulfate was added to the resulting extracted liquid to dehydrate the extracted liquid. Next, the magnesium sulfate was filtered out from the extracted liquid. The resulting filtrate was concentrated by using a rotary evaporator. The resulting concentrated liquid was purified by using silica gel column chromatography, and, accordingly, compound (6)-10, which was a white solid, was obtained. Compound (6)-10 was identified by using ¹H-NMR. FIG. 4 is a graph illustrating the ¹H-NMR spectrum of compound (6)-10. The ¹H-NMR spectrum of compound (6)-10 was as follows: ¹H-NMR (600 MHz, CHLOROFORM-D) δ8.74 (d, J=8.3 Hz, 6 H), 8.05 (d, J=9.0 Hz, 6 H), 7.72 (d, J=8.3 Hz, 6 H), 7.64 (d, J=8.3 Hz, 6 H), 4.33 (t, J=6.5 Hz, 6 H), 1.76-1.81 (m, 6 H), 1.43-1.48 (m, 6 H), 1.27-1.40 (m, 24 H), 0.90 (t, J = 6.9 Hz, 9 H).

### Synthesis of Compound (7)-7

First, 2,4,6-tris(4-bromophenyl)-1,3,5-triazine and 1,3-dimethyl-5-ethynylisophthalate were dissolved in triethylamine. Furthermore, catalytic amounts of triphenylphosphine, bis(triphenylphosphine)palladium(II) dichloride, and copper (I) iodide were added to the resulting solution. Next, the solution was stirred at room temperature for 16 hours. A neutralization process was performed on the resulting reaction solution by adding hydrochloric acid to the resulting reaction solution. Next, an extraction process was performed on the reaction solution by using ethyl acetate. Magnesium sulfate was added to the resulting extracted liquid to dehydrate the extracted liquid. Next, the magnesium sulfate was filtered out from the extracted liquid. The resulting filtrate was concentrated by using a rotary evaporator. The resulting concentrated liquid was purified by using silica gel column chromatography, and, accordingly, a precursor of compound (7)-7 was obtained.

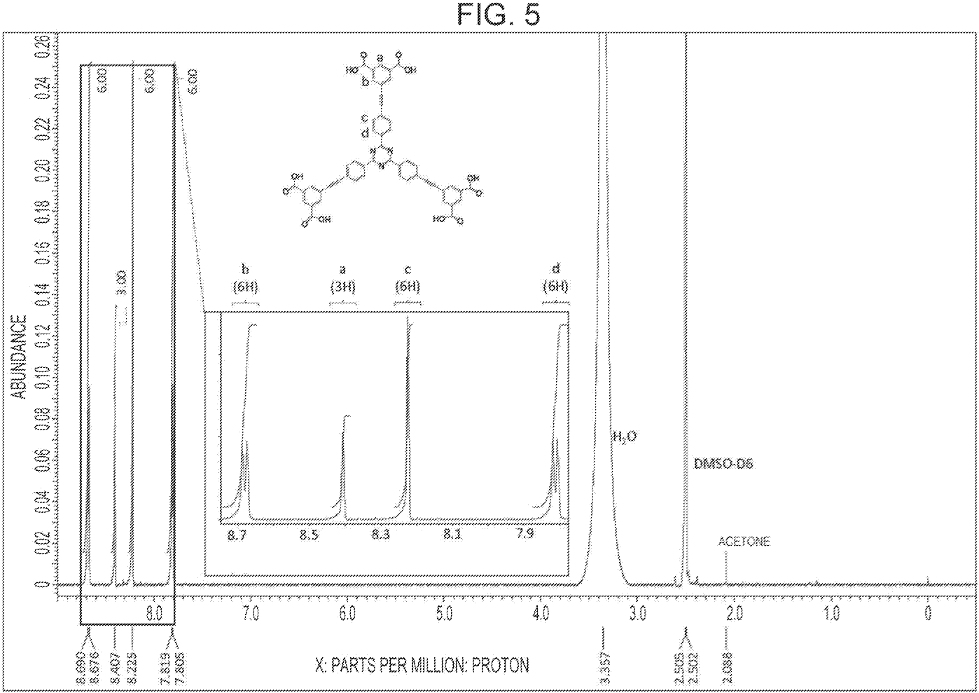

Next, the precursor of compound (7)-7 was dissolved in a liquid mixture containing tetrahydrofuran and methanol (v/v= 1:1). An aqueous sodium hydroxide solution was added to the resulting solution, which was then heated at reflux with stirring overnight. After the reaction in the solution was completed, dilute hydrochloric acid was added to the solution. Accordingly, the solution was acidified, and a solid precipitated out. The solid was washed with purified water, and, accordingly, compound (7)-7, which was a white solid, was obtained. Compound (7)-7 was identified by using ¹H-NMR. FIG. 5 is a graph illustrating the ¹H-NMR spectrum of compound (7)-7. The ¹H-NMR spectrum of compound (7)-7 was as follows: ¹H-NMR (600 MHz, DMSO-D6) 88.68 (d, J=8.3 Hz, 6 H), 8.41 (s, 3 H), 8.22 (s, 6 H), 7.81 (d, J=8.3 Hz, 6 H).

### Measurement of Two-Photon Absorption Cross Section

A measurement of the two-photon absorption cross section with respect to light having a wavelength of 405 nm was performed on the synthesized compounds. The measurement of the two-photon absorption cross section was carried out by using the Z-scan method described in J. Opt. Soc. Am. B, 2003, Vol. 20, p. 529. The light source used to measure the two-photon absorption cross section was a titanium-sapphire pulsed laser. Specifically, a second high frequency wave of the titanium-sapphire pulsed laser was projected onto the samples. The laser had a pulse width of 80 fs. The laser had a repetition frequency of 1 kHz. An average power of the laser was varied over a range of greater than or equal to 0.01 mW and less than or equal to 0.08 mW. The light from the laser was light having a wavelength of 405 nm. Specifically, the light from the laser had a center wavelength of greater than or equal to 402 nm and less than or equal to 404 nm. A full width at half maximum of the light from the laser was 4 nm.

### Estimation of Two-Photon Absorption Cross Section

An estimation of the two-photon absorption cross section with respect to light having a wavelength of 405 nm was performed on the synthesized compounds. Specifically, the two-photon absorption cross section was calculated by performing density functional theory (DFT) calculation in accordance with the secondary non-linear response theory described in J. Chem. Theory Comput 2018, Vol. 14, p. 807. The software used for the DFT calculation was Turbomole version 7.3.1 (available from CosmoLogic Inc.). The basis function used was def2-TZVP. The functional used was B3LYP.

A linear regression was performed on the calculated values and measured values of the two-photon absorption cross section of the synthesized compounds. In the linear regression, an R² value, which is a coefficient of determination, was 0.9. This confirmed a high correlation between the calculated values and measured values of the two-photon absorption cross section. Next, by using a regression equation obtained from the linear regression, calculated values of the two-photon absorption cross section of different compounds were calculated. The different compounds were different from the synthesized compounds in the type of Z.

### Measurement of Quantum Yield of Fluorescence

A measurement of an internal quantum yield of fluorescence was performed on the synthesized compounds. The measurement samples were prepared by dissolving each of the compounds in a dimethyl sulfoxide (DMSO) solvent. An absolute PL quantum yield spectrometer (C9920-02, manufactured by Hamamatsu Photonics K.K.) was used for the measurement. The excitation wavelength was set at 325 nm. The measurement wavelength was adjusted to be within a range of greater than or equal to 350 nm and less than or equal to 650 nm. A DMSO solvent was used as a reference.

### Measurement of Molar Extinction Coefficient

A measurement of the molar extinction coefficient was performed on the synthesized compounds by using a method according to the specifications of JIS K 0115:2004. Specifically, an absorption spectrum of the measurement samples was first measured Absorbance at a wavelength of 405 nm was read from the acquired spectrum. The molar extinction coefficient was calculated based on a concentration of the compound in the measurement sample and an optical path length of the cell used in the measurement Estimation of Molar Extinction Coefficient

An estimation of the molar extinction coefficient was performed on the synthesized compounds. DFT calculation was used to estimate the molar extinction coefficient. Specifically, excited state calculation was first performed on the compounds by using Gaussian 16 (available from Gaussian, Inc.), which is a quantum chemistry calculation program. In the excited state calculation, the basis function used was 6-31++G(d,p). The functional used was CAM-B3LYP. Energy for exciting the compounds and a probability of transition to the excited state were calculated by using the excited state calculation. Furthermore, absorption wavelengths and an oscillator strength f at each of the absorption wavelengths were calculated from these calculation results. The oscillator strength correlates to the molar extinction coefficient. Next, the full width at half maximum was specified assuming that the absorption spectrum had a Gaussian distribution. Specifically, the full width at half maximum was specified to be 0.4 eV, and an absorption spectrum was drawn based on the absorption wavelengths and the oscillator strengths. Absorbance at a wavelength of 405 nm was read from the acquired absorption spectrum. The absorbance was regarded as a calculated value of the molar extinction coefficient.

Tables 2 to 4 show the measured values and calculated values of the two-photon absorption cross section, the quantum yields of fluorescence, and the measured values and calculated values of the molar extinction coefficient, which were obtained by using the methods described above. In Tables 2 to 4, “No Data” means that no data were acquired.

TABLE 2

| | Compound | Two-photon absorption cross section (GM) | | Molar extinction coefficient (L/(mol·cm)) | | Fluorescence quantum yield (%) |
|---|---|---|---|---|---|---|
| | | Measured value | Calculated value | Measured value | Calculated value | |
| Example 1 | (6)-1 | No Data | 430 | No Data | 30 | No Data |
| Example 2 | (6)-2 | No Data | 1400 | No Data | 30 | No Data |
| Example 3 | (6)-3 | No Data | 1620 | No Data | 50 | No Data |
| Example 4 | (6)-4 | No Data | 1690 | No Data | 60 | No Data |
| Example 5 | (6)-5 | No Data | 720 | No Data | 30 | No Data |
| Example 6 | (6)-6 | No Data | 1520 | No Data | 70 | No Data |
| Reference example 1 | (6)-7 | 1780 | 1490 | 70 | 80 | 10 |
| Example 7 | (6)-8 | No Data | 1250 | No Data | 80 | No Data |
| Example 8 | (6)-9 | 1970 | 1740 | 210 | 90 | 10 |
| Example 9 | (6)-10 | 2080 | 1800 | 50 | 90 | 10 |
| Example 10 | (6)-11 | No Data | 1620 | No Data | 100 | No Data |
| Example 11 | (6)-12 | No Data | 1640 | No Data | 70 | No Data |
| Example 12 | (6)-13 | No Data | 1440 | No Data | 70 | No Data |
| Reference example 2 | (6)-14 | No Data | 1070 | No Data | 90 | No Data |
| Example 13 | (6)-15 | No Data | 1730 | No Data | 45 | No Data |
| Example 14 | (6)-16 | No Data | 1700 | No Data | 115 | No Data |
| Example 15 | (6)-17 | No Data | 1640 | No Data | 45 | No Data |
| Example 16 | (6)-18 | No Data | 1810 | No Data | 60 | No Data |

TABLE 3

| | Compound | Two-photon absorption cross section (GM) | | Molar extinction coefficient (L/(mol·cm)) | | Fluorescence quantum yield (%) |
|---|---|---|---|---|---|---|
| | | Measured value | Calculated value | Measured value | Calculated value | |
| Example 17 | (6)-19 | No Data | 1700 | No Data | 45 | No Data |
| Example 18 | (6)-20 | No Data | 1740 | No Data | 50 | No Data |
| Example 19 | (6)-2 1 | No Data | 1500 | No Data | 190 | No Data |
| Example 20 | (6)-22 | No Data | 1410 | No Data | 445 | No Data |
| Example 21 | (6)-23 | No Data | 1300 | No Data | 140 | No Data |
| Example 22 | (6)-24 | No Data | 660 | No Data | 10 | No Data |
| Example 23 | (7)-7 | 1360 | No Data | 40 | 25 | 10 |
| Example 24 | (9)-1 | No Data | 1980 | No Data | 65 | No Data |
| Example 25 | (9)-2 | No Data | 1870 | No Data | 60 | No Data |
| Example 26 | (9)-3 | No Data | 2260 | No Data | 100 | No Data |
| Example 27 | (9)-4 | No Data | 2360 | No Data | 105 | No Data |
| Example 28 | (9)-5 | No Data | 2530 | No Data | 60 | No Data |
| Example 29 | (9)-6 | No Data | 1910 | No Data | 125 | No Data |
| Example 30 | (9)-7 | No Data | 2700 | No Data | 125 | No Data |
| Example 31 | (9)-8 | No Data | 2870 | No Data | 130 | No Data |
| Example 32 | (9)-9 | No Data | 3050 | No Data | 135 | No Data |
| Example 33 | (9)-10 | No Data | 3140 | No Data | 140 | No Data |

TABLE 4

| | Compound | Two-photon absorption cross section (GM) | | Molar extinction coefficient (L/(mol·cm)) | | Fluorescence quantum yield (%) |
|---|---|---|---|---|---|---|
| | | Measured value | Calculated value | Measured value | Calculated value | |
| Example 34 | (9)-11 | No Data | 2650 | No Data | 150 | No Data |
| Example 35 | (9)-12 | No Data | 2730 | No Data | 105 | No Data |
| Example 36 | (9)-13 | No Data | 2800 | No Data | 110 | No Data |
| Example 37 | (9)-14 | No Data | 2370 | No Data | 165 | No Data |
| Example 38 | (9)-15 | No Data | 2680 | No Data | 105 | No Data |
| Example 39 | (9)-16 | No Data | 2350 | No Data | 185 | No Data |
| Example 40 | (9)-17 | No Data | 2820 | No Data | 80 | No Data |
| Example 41 | (9)-18 | No Data | 2950 | No Data | 105 | No Data |
| Example 42 | (9)-19 | No Data | 2950 | No Data | 75 | No Data |
| Example 43 | (9)-20 | No Data | 3000 | No Data | 80 | No Data |
| Example 44 | (9)-21 | No Data | 1650 | No Data | 340 | No Data |
| Example 45 | (9)-22 | No Data | 1690 | No Data | 780 | No Data |
| Example 46 | (9)-23 | No Data | 2130 | No Data | 185 | No Data |
| Example 47 | (9)-24 | No Data | 2280 | No Data | 110 | No Data |

Next, compounds different from the compound A represented by formula (1) were prepared as listed in Table 5 below. Note that compound If, which is the compound of Comparative Example 3, is represented by formula (13) below.

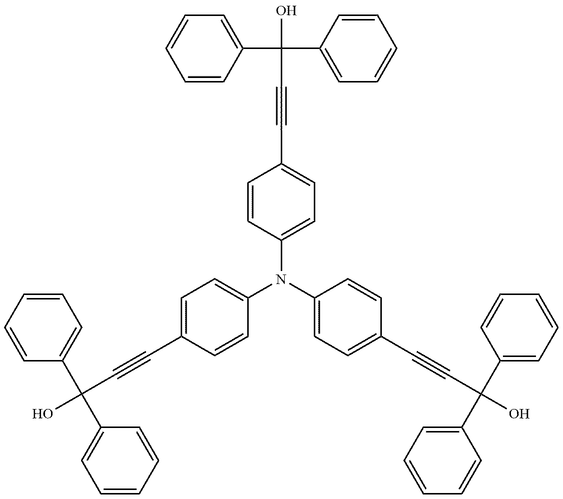 [SMILES: OC(C#Cc1ccc(N(c2ccc(C#CC(O)(c3ccccc3)c3ccccc3)cc2)c2ccc(C#CC(O)(c3ccccc3)c3ccccc3)cc2)cc1)(c1ccccc1)c1ccccc1]

Next, measurements of the two-photon absorption cross section, the molar extinction coefficient, and the quantum yield of fluorescence of the compounds listed in Table 5 were performed by using the methods described above. For the 2,4,6-tris(4-carboxyphenyl)-s-triazine of Comparative Example 1, an estimation of the two-photon absorption cross section was also performed. The results are shown in Table 5. In Table 5, “No Data” means that no data were acquired.

TABLE 5

| | Compound | Two-photon absorption cross section (GM) | | Molar extinction coefficient (L/(mol·cm)) | | Fluorescence quantum yield (%) |
|---|---|---|---|---|---|---|
| | | Measured value | Calculated value | Measured value | Calculated value | |
| Comparative example 1 | 2,4,6-tris(4-carboxyphcnvl)-s-triazine (H₃TATB) | 30 | 20 | 0 | No Data | 0 |
| Comparative example 2 | hexakis(phenylethynyl)benzene (HPEB) | 23000 | No Data | 4010 | No Data | 10 or greater and 30 or less |
| Comparative example 3 | 1f | 380 | No Data | 70 | No Data | 30 |
| Comparative example 4 | 4-fluoro-4'-(phenylethnyl)-benzophenone | 120 | No Data | 0 | No Data | No Data |
| Comparative example 5 | 1,1,3-triplietty 1-2-propyn-1 -ol | 0 | No Data | 100 | No Data | No Data |
| Comparative example 6 | 1,4-diphenylbutadiyne | 410 | No Data | 0 | No Data | No Data |
| Comparative example 7 | benzo[h]quinoline | 10 | No Data | 0 | No Data | No Data |
| Comparative example 8 | 2-isopropenylnaphthalene | 10 | No Data | 0 | No Data | No Data |

As can be seen from Tables 2 to 4, all of the compounds of Examples 1 to 47, which correspond to the compound A represented by formula (1), had a two-photon absorption cross section of greater than 410 GM with respect to light having a wavelength of 405 nm. In addition, the compounds of Examples 1 to 47 had a molar extinction coefficient of less than or equal to 800 L/(mol · cm) with respect to light having a wavelength of 405 nm. These results demonstrate that the compounds of Examples 1 to 47 have two-photon absorption properties in which high non-linearity is exhibited with respect to light having a wavelength in a short wavelength range.

The hexakis(phenylethynyl)benzene of Comparative Example 2, which is a hexasubstituted benzene, had a large value of the two-photon absorption cross section with respect to light having a wavelength of 405 nm and also had a significantly large value of the molar extinction coefficient of 4010 L/(mol · cm). In hexasubstituted benzenes, compared with trisubstituted benzenes, the single-photon absorption peak tends to shift to a longer wavelength range because of the extension of the π electron conjugated system. As a result, presumably, the molar extinction coefficient at 405 nm increased in the compound of Comparative Example 2. The compound A represented by formula (1) has a trisubstituted triazine ring and also has an extended π electron conjugated system. Because of this structure, presumably, the compound A has two-photon absorption properties in which high non-linearity is exhibited.

In addition, all of the compounds (6)-7, (6)-9, (6)-10, and (7)-7 had a quantum yield of fluorescence of 10%. This demonstrates that the compound A represented by formula (1) tends to emit fluorescent light in instances in which the compound A absorbs excitation light.

Compounds or non-linear optical materials of the present disclosure can be used, for example, in applications for a recording layer of a three-dimensional optical memory, a photocurable resin composition for stereolithography, and the like. The compounds or the non-linear optical materials of the present disclosure tend to have two-photon absorption properties in which high non-linearity is exhibited with respect to light having a wavelength in a short wavelength range. Accordingly, the compounds or the non-linear optical materials of the present disclosure enable realization of very high spatial resolution in applications such as in three-dimensional optical memories and additive manufacturing apparatuses. In addition, the compounds or the non-linear optical materials of the present disclosure tend to emit fluorescent light. Accordingly, using any of the compounds or the non-linear optical materials in a recording layer of a three-dimensional optical memory makes it possible to employ a method of reading the ON/OFF state of the recording layer based on changes in the fluorescence from the compound or the non-linear optical material. It is also possible that the compounds or the non-linear optical materials of the present disclosure can be used in fluorochrome materials that are used, for example, in two-photon fluorescence microscopes.

What is claimed is:

1. A non-linear optical material represented by formula (1) below, 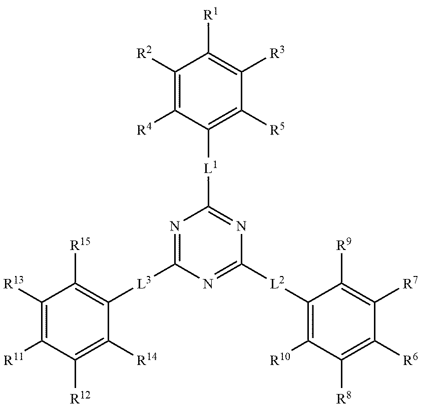
where R¹ to R¹³ each independently include at least one atom selected from the group consisting of H, C, N, O, F, P, S, Cl, I, and Br, and L¹ to L³ are each independently represented by formula (2) or (3) below, 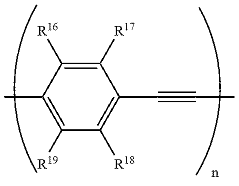 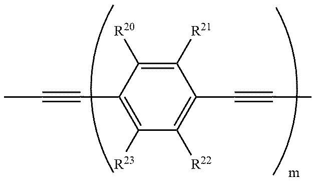
where
R¹⁶ to R¹⁹ each independently include at least one atom selected from the group consisting of H, C, N, O, F, P, S, Cl, I, and Br, and n is an integer of 1 to 3, and
R²⁰ to R²³ each independently include at least one atom selected from the group consisting of H, C, N, O, F, P, S, Cl, I, and Br, and m is an integer of 1 to 3,
wherein, when the non-linear optical material is represented by formula (4) below, 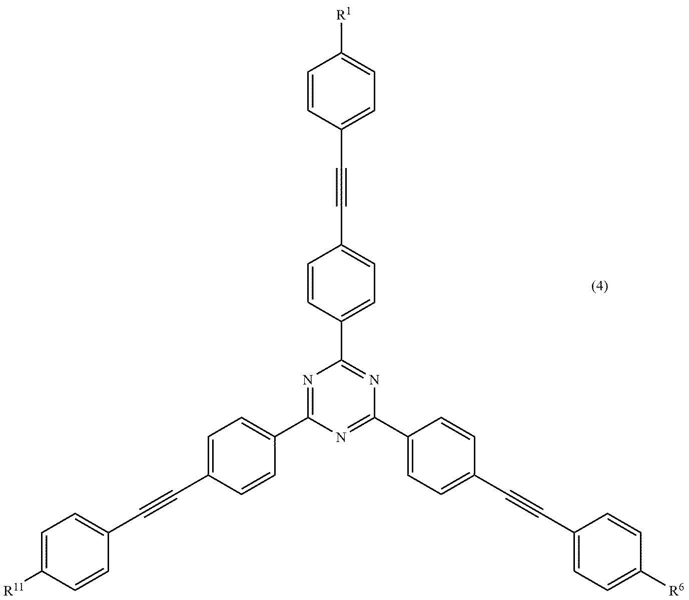
at least one selected from the group consisting of R¹, R⁶, and R¹¹ is a halogen atom, a halogenated alkyl group, an unsaturated hydrocarbon group, a hydroxyl group, an alkoxycarbonyl group, an acyl group, an amide group, an acyloxy group, a thiol group, an alkylthio group, a sulfonic acid group, an acylthio group, an alkylsulfonyl group, a sulfonamide group, a primary amino group, a secondary amino group, or a nitro group.

2. The non-linear optical material according to claim 1, wherein the non-linear optical material is represented by formula (5) below, 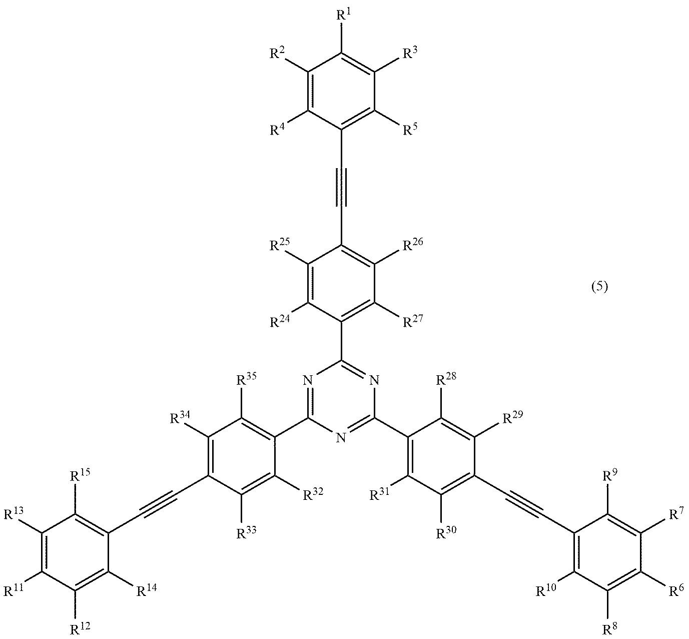
where R²⁴ to R³⁵ each independently include at least one atom selected from the group consisting of H, C, N, O, F, P, S, Cl, I, and Br.

3. The non-linear optical material according to claim 1, wherein R¹ to R¹⁵ are each independently a hydrogen atom, a halogen atom, an alkyl group, a halogenated alkyl group, an unsaturated hydrocarbon group, a hydroxyl group, a carboxyl group, an alkoxycarbonyl group, an acyl group, an amide group, a nitrile group, an alkoxy group, an acyloxy group, a thiol group, an alkylthio group, a sulfonic acid group, an acylthio group, an alkylsulfonyl group, a sulfonamide group, a primary amino group, a secondary amino group, a tertiary amino group, or a nitro group.

4. The non-linear optical material according to claim 1, wherein at least one selected from the group consisting of R¹ to R³, R⁶ to R⁸, and R¹¹ to R¹³ is an electron-donating group or an electron-withdrawing group.

5. The non-linear optical material according to claim 1, wherein at least one selected from the group consisting of R¹ to R³, R⁶ to R⁸, and R¹¹ to R¹³ is an alkoxycarbonyl group.

6. The non-linear optical material according to claim 1, wherein at least one selected from the group consisting of R¹ to R³, R⁶ to R⁸, and R¹¹ to R¹³ is —COOC₄H₉ or —COOC₈H₁₇.

7. A compound that is used in a device that utilizes light having a wavelength of greater than or equal to 390 nm and less than or equal to 420 nm, the compound being represented by formula (1) below, 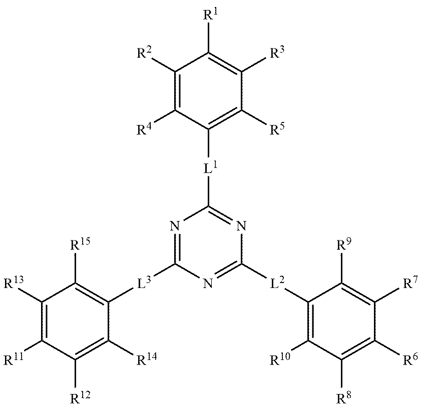
where R¹ to R¹⁵ each independently include at least one atom selected from the group consisting of H, C, N, O, F, P, S, Cl, I, and Br, and L¹ to L³ are each independently represented by formula (2) or (3) below, 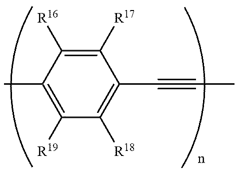 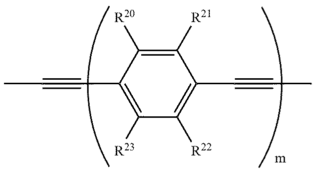
where
R¹⁶ to R¹⁹ each independently include at least one atom selected from the group consisting of H, C, N, O, F, P, S, Cl, I, and Br, and n is an integer of 1 to 3, and
R²⁰ to R²³ each independently include at least one atom selected from the group consisting of H, C, N, O, F, P, S, Cl, I, and Br, and m is an integer of 1 to 3.

8. , A recording medium comprising a non-linear optical material represented by formula (1) below, 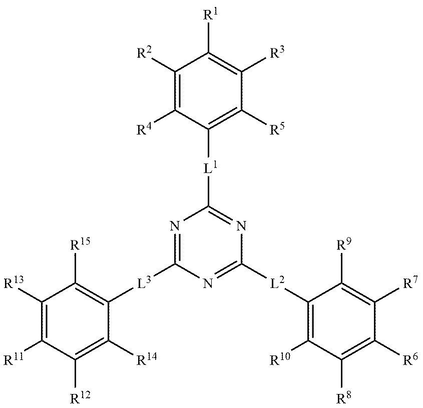
where R¹ to R¹⁵ each independently include at least one atom selected from the group consisting of H, C, N, O, F, P, S, Cl, I, and Br, and L¹ to L³ are each independently represented by formula (2) or (3) below, 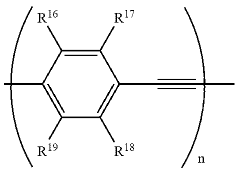 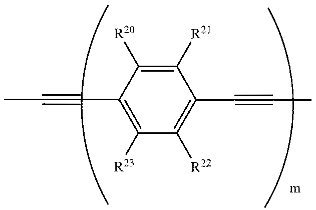
where
R¹⁶ to R¹⁹ each independently include at least one atom selected from the group consisting of H, C, N, O, F, P, S, Cl, I, and Br, and n is an integer of 1 to 3, and
R²⁰ to R²³ each independently include at least one atom selected from the group consisting of H, C, N, O, F, P, S, Cl, I, and Br, and m is an integer of 1 to 3.

9. A method for recording information, the method comprising:
providing a light source that emits light having a wavelength of greater than or equal to 390 nm and less than or equal to 420 nm; and
focusing the light from the light source and projecting the light onto a recording region of a recording medium that includes a compound represented by formula (1) below, 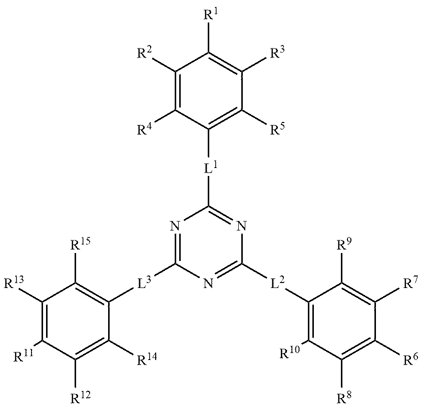
where R¹ to R¹³ each independently include at least one atom selected from the group consisting of H, C, N, O, F, P, S, Cl, I, and Br, and L¹ to L³ are each independently represented by formula (2) or (3) below, 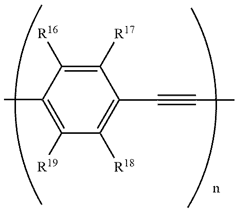 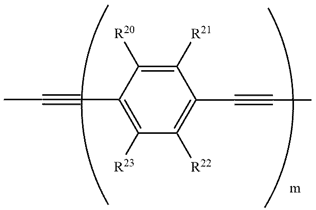
where
R¹⁶ to R¹⁹ each independently include at least one atom selected from the group consisting of H, C, N, O, F, P, S, Cl, I, and Br, and n is an integer of 1 to 3, and
R²⁰ to R²³ each independently include at least one atom selected from the group consisting of H, C, N, O, F, P, S, Cl, I, and Br, and m is an integer of 1 to 3.

10. A method for reading information recorded by the method according to claim 9, the method for reading information comprising:
measuring an optical property of the recording region by projecting light onto the recording region of the recording medium; and
determining, based on the optical property, whether information is recorded in the recording region.

11. The method according to claim 10, wherein the optical property is an intensity of light that reflects off the recording region.
